SEQUENCE LISTING (2 sequences)

SEQ ID NO: 1 - protein, 563 aa, Homo sapiens
MGLQACLLGLFALILSGKCSYSPEPDQRRTLPPGWVSLGRADPEEELSLTFALRQQNVERLSELVQAVSDPSSPQYGKYLTLENVADLVRPSPLTLHTVQKWLLAAGAQKCHSVITQDFLTCWLSIRQAELLLPGAEFHHYVGGPTETHVVRSPHPYQLPQALAPHVDFVGGLHRFPPTSSLRQRPEPQVTGTVGLHLGVTPSVIRKRYNLTSQDVGSGTSNNSQACAQFLEQYFHDSDLAQFMRLFGGNFAHQASVARVVGQQGRGRAGIEASLDVQYLMSAGANISTWVYSSPGRHEGQEPFLQWLMLLSNESALPHVHTVSYGDDEDSLSSAYIQRVNTELMKAAARGLTLLFASGDSGAGCWSVSGRHQFRPTFPASSPYVTTVGGTSFQEPFLITNEIVDYISGGGFSNVFPRPSYQEEAVTKFLSSSPHLPPSSYFNASGRAYPDVAALSDGYWVVSNRVPIPWVSGTSASTPVFGGILSLINEHRILSGRPPLGFLNPRLYQQHGAGLFDVTRGCHESCLDEEVEGQGFCSGPGWDPVTGWGTPNFPALLKTLLNP

SEQ ID NO: 2 - DNA, 1692 bp, Homo sapiens
  source 1..1692: mol_type = genomic DNA
atgggactccaagcctgcctcctagggctctttgccctcatcctctctggcaaatgcagttacagcccggagcccgaccagcggaggacgctgcccccaggctgggtgtccctgggccgtgcggaccctgaggaagagctgagtctcacctttgccctgagacagcagaatgtggaaagactctcggagctggtgcaggctgtgtcggatcccagctctcctcaatacggaaaatacctgaccctagagaatgtggctgatctggtgaggccatccccactgaccctccacacggtgcaaaaatggctcttggcagccggagcccagaagtgccattctgtgatcacacaggactttctgacttgctggctgagcatccgacaagcagagctgctgctccctggggctgagtttcatcactatgtgggaggacctacggaaacccatgttgtaaggtccccacatccctaccagcttccacaggccttggccccccatgtggactttgtggggggactgcaccgttttcccccaacatcatccctgaggcaacgtcctgagccgcaggtgacagggactgtaggcctgcatctgggggtaaccccctctgtgatccgtaagcgatacaacttgacctcacaagacgtgggctctggcaccagcaataacagccaagcctgtgcccagttcctggagcagtatttccatgactcagacctggctcagttcatgcgcctcttcggtggcaactttgcacatcaggcatcagtagcccgtgtggttggacaacagggccggggccgggccgggattgaggccagtctagatgtgcagtacctgatgagtgctggtgccaacatctccacctgggtctacagtagccctggccggcatgagggacaggagcccttcctgcagtggctcatgctgctcagtaatgagtcagccctgccacatgtgcatactgtgagctatggagatgatgaggactccctcagcagcgcctacatccagcgggtcaacactgagctcatgaaggctgccgctcggggtctcaccctgctcttcgcctcaggtgacagtggggccgggtgttggtctgtctctggaagacaccagttccgccctaccttccctgcctccagcccctatgtcaccacagtgggaggcacatccttccaggaacctttcctcatcacaaatgaaattgttgactatatcagtggtggtggcttcagcaatgtgttcccacggccttcataccaggaggaagctgtaacgaagttcctgagctctagcccccacctgccaccatccagttacttcaatgccagtggccgtgcctacccagatgtggctgcactttctgatggctactgggtggtcagcaacagagtgcccattccatgggtgtccggaacctcggcctctactccagtgtttggggggatcctatccttgatcaatgagcacaggatccttagtggccgcccccctcttggctttctcaacccaaggctctaccagcagcatggggcaggactctttgatgtaacccgtggctgccatgagtcctgtctggatgaagaggtagagggccagggtttctgctctggtcctggctgggatcctgtaacaggctggggaacacccaacttcccagctttgctgaagactctactcaacccctga

(19) United States
(12) Patent Application Publication
(10) Pub. No.: US 2025/0338833 A1
(43) Pub. Date: Nov. 6, 2025

(54) METHODS FOR TREATING LYSOSOMAL STORAGE DISEASES

(71) Applicant: RUTGERS, THE STATE UNIVERSITY OF NEW JERSEY, New Brunswick, NJ (US)

(72) Inventors: David Sleat, Scotch Plains, NJ (US); Peter Lobel, Highland Park, NJ (US)

(21) Appl. No.: 18/854,132

(22) PCT Filed: Apr. 4, 2023
(86) PCT No.: PCT/US23/65318

§ 371 Date: Oct. 4, 2024

Related U.S. Application Data
(60) Provisional application No. 63/362,574, filed on Apr. 6, 2022.

(51) Int. Cl. A01K 67/0276; A61K 38/48; A61K 49/00

(52) U.S. Cl. CPC A01K 67/0276; A61K 38/4813; A61K 49/0008; C12Y 304/14009; A01K 2217/075; A01K 2227/105; A01K 2267/0306

(57) ABSTRACT

This disclosure provides novel animal models for use in studying lysosomal storage diseases and methods of treating lysosomal storage diseases by increasing a level or activity of TPP1.

### CROSS-REFERENCE TO RELATED APPLICATIONS

This application claims priority under 35 U.S.C. § 119(e) to U.S. Provisional Patent Application No. 63/362,574, filed Apr. 6, 2022. The foregoing application is incorporated by reference herein in its entirety.

### FIELD OF THE INVENTION

The present disclosure relates generally to methods of treating lysosomal storage diseases.

### BACKGROUND OF THE INVENTION

The neuronal ceroid lipofuscinoses (NCLs) are a group of more than a dozen genetically distinct but similar lysosomal storage diseases. Characterized by an accumulation of autofluorescent storage material within the lysosome, NCLs are neurodegenerative and progressive diseases that are manifested by seizures, loss of vision, and eventual loss of mental capacity. Onset is typically in childhood, and these diseases result in premature death. Two of the most frequently encountered NCL diseases are the late-infantile and juvenile forms (LINCL and JNCL, respectively). LINCL results from mutations in the gene encoding tripeptidyl peptidase 1 (TPP1, formerly designated CLN2), a soluble lysosomal serine protease. Disease in LINCL typically presents at around 4 years of age, and lifespan is ˜8 to 15 years. JNCL is caused by mutations in a gene encoding a lysosomal transmembrane protein, CLN3. JNCL has a later onset and is more slowly progressing than LINCL, with initial signs of disease (such as problems with vision) at around 8 years and patients frequently surviving into the second or third decade of life. Despite differences in disease timeline and genetic etiology, LINCL and JNCL have a number of similarities, including lysosomal storage of subunit c of mitochondrial ATP synthase (SCMAS).

There is considerable focus on the development of effective therapies for NCL diseases, and LINCL is leading the way. Enzyme replacement therapy has been clinically approved for LINCL, and there is interest in gene therapy, with promising results obtained in animal models, and clinical studies have been conducted. Both enzyme replacement therapy (ERT) and gene therapy rely upon the fact that TPP1 is a soluble lysosomal protein. In ERT, exogenously-administered recombinant protein can be taken up by numerous cells by endocytosis and delivered to the lysosome, while in gene therapy, only a proportion of cells are transduced but these can overproduce and secrete enzyme that is taken up by untransduced cells. In addition, LINCL animal models with well-defined phenotypes that recapitulate the human disease have been integral in testing treatment strategies.

There is currently no approved therapy for JNCL, and this reflects several major obstacles. First, CLN3 is a transmembrane protein, which excludes replacement treatment using exogenously administered recombinant protein. There is interest in gene therapy for JNCL, but because CLN3 is an integral membrane protein, non-transduced cells will not express the missing protein. If the underlying metabolic defect is cell-autonomous, cross-protection between transduced and untransduced cells may not be possible, requiring a very high proportion of cells to be transduced for effective therapy. Second, there is a fundamental lack of understanding of the cellular function of the CLN3 protein. It has been implicated in numerous cellular activities, including lysosomal pH homeostasis, endocytosis, autophagy, apoptosis, lysosomal enzyme transport, and others, but its precise function is yet to be definitively established. As a result, mechanism-based approaches to treatment for JNCL are currently not an option. Third, animal models for JNCL do not present a robust phenotype, especially with respect to survival. There are several mouse models for JNCL, and their phenotypes are very similar, but the disease is highly attenuated compared to mouse models of other NCL diseases.

Accordingly, there remains a need for animal models for use in studying lysosomal storage diseases and methods of treating lysosomal storage diseases.

### SUMMARY OF THE INVENTION

In one aspect, this disclosure provides a method of treating a subject having a disease or disorder characterized by accumulation of SCMAS in the lysosomes of affected cells. In some embodiments, the method comprises administering to the subject a therapeutically effective amount of an agent that increases a level or activity of TPP1, to reduce or eliminate symptoms caused by the disease or disorder.

In some embodiments, the disease or disorder is selected from Juvenile neuronal ceroid lipofuscinosis (CLN3) disease, Variant late infantile neuronal ceroid lipofuscinosis type 5 (CLN5) disease, Variant late infantile neuronal ceroid lipofuscinosis type 6 (CLN6) disease, Neuronal ceroid lipofuscinosis type 7 (CLN7) disease, Northern epilepsy neuronal ceroid lipofuscinosis type 8 (CLN8) disease, Congenital neuronal ceroid lipofuscinosis type 10 (CLN10) disease, Late-onset Neuronal ceroid lipofuscinosis (CLN12) and Kufor-Rakeb syndrome, Sanfilippo D syndrome (mucopolysaccharidosis type IIID), and Osteopetrosis autosomal recessive 4 (OPTB4).

In some embodiments, the disease or disorder is characterized by a deficiency in a function of a CLN3 protein.

In some embodiments, the affected cells are neuronal cells. In some embodiments, the affected cells are in a tissue or organ, such as liver, spleen, or brain.

In some embodiments, the agent reduces the level of accumulation of the SCMAS in the lysosomes of the affected cells. In some embodiments, the agent comprises a recombinant human TPP1 protein. In some embodiments, the TPP1 protein is an inactive proenzyme. In some embodiments, the TPP1 protein is mannose-6-phosphorylated.

In some embodiments, the therapeutically effective amount of the TPP1 protein is such that the affected cells receive from about 1.0 to about 100 nM of recombinant human TPP1 protein.

In some embodiments, the agent comprises a nucleic acid molecule comprising a nucleotide sequence encoding TPP1 or a variant thereof.

In some embodiments, the agent is administered by injection. In some embodiments, the injection is intracranial. In some embodiments, the agent is delivered to lysosomes of the affected cells. In some embodiments, the agent is administered in a controlled release system.

In some embodiments, the subject is a mammal, e.g., a human.

In another aspect, this disclosure provides an animal model for studying a disease or disorder, such as a lysosomal storage disease. In some embodiments, the animal model comprises: (i) a tripeptidyl peptidase 1 (Tpp1) gene heterozygous knockout (Tpp1^{+/-}), and (ii) a Cln3 gene homozygous knockout (Cln3^{-/-}), wherein the mouse model has a shortened lifespan compared to a wild type animal. In some embodiments, the animal is a mouse.

In some embodiments, the disease or disorder is selected from Late-infantile neuronal ceroid lipofuscinosis (CLN2) disease, Juvenile neuronal ceroid lipofuscinosis (CLN3) disease, Variant late infantile neuronal ceroid lipofuscinosis type 5 (CLN5) disease, Variant late infantile neuronal ceroid lipofuscinosis type 6 (CLN6) disease, Neuronal ceroid lipofuscinosis type 7 (CLN7) disease, Northern epilepsy neuronal ceroid lipofuscinosis type 8 (CLN8) disease, Congenital neuronal ceroid lipofuscinosis type 10 (CLN10) disease, Late-onset Neuronal ceroid lipofuscinosis (CLN12) and Kufor-Rakeb syndrome, Sanfilippo D syndrome (mucopolysaccharidosis type IIID), and Osteopetrosis autosomal recessive 4 (OPTB4).

In some embodiments, the animal has at least 25% reduction in lifespan compared to the wild type animal.

In some embodiments, the Tpp1 gene or the Cln3 gene comprises at least one mutation selected from a deletion, an insertion, a frame-shift mutation, re-arrangement or a substitution. In some embodiments, the mutation is constitutive. In some embodiments, the mutation is conditional.

In some embodiments, the Tpp1 gene is located at Chr 7 E3; 7 55.97 cM. In some embodiments, the Tpp1 gene comprises a deletion of at least a portion of an exon within the Tpp1 gene. In some embodiments, the Tpp1 gene comprises an insertion of neo into intron 11 and an Arg446His missense mutation into exon 11 immediately upstream of the neo insertion.

In some embodiments, wherein the Cln3 gene is located at Chr 7 F3; 7 69.16 cM. In some embodiments, the Cln3 gene comprises a deletion of at least a portion of an exon within the Cln3 gene. In some embodiments, the Cln3 gene comprises a deletion of all or part of exons 1-6 within the Cln3 gene.

In some embodiments, the animal model has an increased level of lysosomal accumulation of subunit c of mitochondrial ATP synthase (SCMAS). In some embodiments, the animal model has at least 50% increase in the level of lysosomal accumulation of SCMAS.

In some embodiments, the animal model has an increased expression level of Niemann-Pick disease type C1 (NPC1) and/or Cathepsin F (CTSF). In some embodiments, the animal model has at least 40% increase in the expression level of NPC1 and/or CTSF, or 40% decrease in the expression level of acid sphingomyelinase (SMPD1).

In some embodiments, the animal model is characterized by a deficit in a locomotor activity.

Also within the scope of this disclosure is a progeny of the animal model disclosed herein, and a cell, tissue, or cell line derived from the animal model or the progeny, as disclosed herein. In another aspect, this disclosure also provides a method of obtaining the animal model disclosed above. The method comprises: (a) cross-breeding an animal with a Tpp1 knockout with a second animal with a Cln3 knockout to obtain an animal with double heterozygotes (Tpp1^{+/-}; Cln3^{+/-}); and (b) cross-breeding the animal with double heterozygotes by mating Tpp1^{-/-}; Cln3^{-/-}×Tpp1^{-/-}; Cln3^{-/-} or Tpp1^{-/-}; Cln3^{-/+}×Tpp1^{+/-}; Cln3^{-/-}.

In another aspect, this disclosure further provides a method of identifying an agent for use in treatment of a disease or disorder in a subject. In some embodiments, the method comprises administering a candidate agent to the animal model or the progeny, as disclosed herein, and assessing an effect of the candidate agent on a phenotype of the animal model. In some embodiments, the method comprises contacting the cell, tissue, or cell line, as disclosed herein, with a candidate agent, and assessing an effect of the candidate agent on the cell, tissue, or cell line.

In some embodiments, the disease or disorder is characterized by accumulation of SCMAS in the lysosomes of affected cells (e.g., neuronal cells).

In some embodiments, the disease or disorder is selected from Late-infantile neuronal ceroid lipofuscinosis (CLN2) disease, Juvenile neuronal ceroid lipofuscinosis (CLN3) disease, Variant late infantile neuronal ceroid lipofuscinosis type 5 (CLN5) disease, Variant late infantile neuronal ceroid lipofuscinosis type 6 (CLN6) disease, Neuronal ceroid lipofuscinosis type 7 (CLN7) disease, Northern epilepsy neuronal ceroid lipofuscinosis type 8 (CLN8) disease, Congenital neuronal ceroid lipofuscinosis type 10 (CLN10) disease, Late-onset Neuronal ceroid lipofuscinosis (CLN12) and Kufor-Rakeb syndrome, Sanfilippo D syndrome (mucopolysaccharidosis type IIID), and Osteopetrosis autosomal recessive 4 (OPTB4).

In some embodiments, the phenotype is the lifespan of the animal model.

In some embodiments, the effect is characterized by an increase in the lifespan of the animal model. In some embodiments, the effect is characterized by a decrease in the level of lysosomal accumulation of SCMAS. In some embodiments, the effect is characterized by a decrease in the expression level of NPC1 and/or CTSF.

In some embodiments, the candidate agent comprises a protein, a peptide, a peptidomimetic, a nucleic acid, or a small molecule.

The foregoing summary is not intended to define every aspect of the disclosure, and additional aspects are described in other sections, such as the following detailed description. The entire document is intended to be related as a unified disclosure, and it should be understood that all combinations of features described herein are contemplated, even if the combination of features are not found together in the same sentence, or paragraph, or section of this document. Other features and advantages of the invention will become apparent from the following detailed description. It should be understood, however, that the detailed description and the specific examples, while indicating specific embodiments of the disclosure, are given by way of illustration only, because various changes and modifications within the spirit and scope of the disclosure will become apparent to those skilled in the art from this detailed description.

### BRIEF DESCRIPTION OF THE DRAWINGS

FIG. 1 shows the results of survival analysis of mice in a Tpp1^{-/-} background. Data were obtained from ˜equal numbers of male and female littermates from matings between Tpp1 and Cln3 mutants. Historical data for Tpp1^{-/-}; Cln3^{+/+} are included for animals in a mixed C57BL/6N, C57BL/6J genetic background: survival did not differ significantly from littermates of the same genotype from matings between Tpp1 and Cln3 mutants (Log-rank P=0.8475. Median survival and number of animals analyzed per genotype are shown in Table 1.

FIGS. 2A and 2B show the results of survival analysis of mice in a Tpp1^{+/-} or Tpp1^{+/+} background. FIG. 2A shows a comparison of Tpp1Cln3 mutant mice. Data were obtained from equal numbers of male and female animals. Median survival and number of animals analyzed per genotype are shown in Table 1. The Yuan survival dataset (Yuan R, et al. (2012) Proc Natl Acad Sci USA 109:8224-9) was obtained from Jackson Laboratories (https://phenome.jax.org/projects/Yuan2) and comprised male and female mice in a C57BL/6J substrain background. FIG. 2B shows a comparison of survival of Tpp1^{+/+}; Cln3^{-/-} animals from the current study with historical data for wild type and Tpp1^{+/+}; Cln3^{-/-} animals.

FIG. 3 shows similar levels of astrocytosis in Tpp1^{-/-}Cln3^{+/+} and Tpp1^{-/-}Cln3^{-/-} animals. Immunofluorescence staining for the astrocyte marker glial fibrillary associated protein (GFAP) reveals profound astrocytosis in the primary somatosensory cortex (S1BF) and ventral posterior thalamic nuclei (VPM/VPL) of both Tpp1^{-/-}Cln3^{+/+} and Tpp1^{-/-}Cln3^{-/-} animals, compared to the relatively low level of GFAP immunoreactivity in the grey matter of Tpp1^{-/-}Cln3^{-/-} and Tpp1^{+/-} Cln^{+/-}, which is comparable to previously published data from wild type mice in which GFAP staining is largely confined to the white matter. Inserts reveal the corresponding astrocytic hypertrophy in Tpp1^{-/-}Cln3^{+/+} and Tpp1^{-/-}Cln3^{-/-} mice. Scale bar=100 μm and 25 μm in inserts.

FIG. 4 shows similar levels of microglial activation in Tpp1^{-/-}Cln3^{+/+} and Tpp1^{-/-}Cln3^{-/-} animals. Immunofluorescence staining for the microglial marker CD68 reveals profound microglial activation in the primary somatosensory cortex (S1BF) and ventral posterior thalamic nuclei (VPM/VPL) of both Tpp1^{-/-}Cln3^{+/+} and Tpp1^{-/-}Cln3 ^{/-} animals, compared to the relatively low level of CD68 immunoreactivity in Tpp1^{+/-}Cln3^{-/-} and Tpp1^{+/-}/Cln3^{+/-}, which is comparable to previously published data from wild type mice. Inserts reveal the corresponding microglial hypertrophy in Tpp1^{-/-}Cln3^{+/+} and Tpp1^{-/-}Cln3^{-/-} mice. Scale bar=100 μm and 25 μm in inserts.

FIG. 5 shows the results of a quantitative analysis of glial activation. Thresholding image analysis confirms the different levels of glial fibrillary associated protein (GFAP, astrocytes) and CD68 (microglia) in the primary somatosensory cortex (S1BF) and ventral posterior thalamic nuclei (VPM/VPL) of animals of different genotypes. These data confirm the significantly elevated levels of both antigens in Tpp1^{-/-}Cln3^{+/+} and Tpp1^{-/-}Cln3^{-/-} mice compared to animals of other genotypes, which displayed very low levels of immunoreactivity for these markers. Asterisk indicates adjusted p value of <0.05 when compared to Tpp1^{+/+}; Cln3^{+/-} by one-way ANOVA using Dunnett's test for multiple comparisons.

FIG. 6 shows relative protein expression in Tpp1 and Cln3 mutant mice determined by SPS-MS3 quantitation of isobaric-labeled peptides. Q values were calculated based on peptide data for each protein using the False Discovery Rate two-stage step-up method of Benjamini, Krieger, and Yekutieli. Dashed lines indicate an FDR of 1% (y axis) and arbitrary fold-change of 2-fold (x axis). Axes are truncated at Q value 1E-10 and ratios of 1/16 to 16. Red and black symbols indicate known lysosomal (based on (17)) and other proteins, respectively. Names of select proteins of interest are shown in red (lysosomal) or black (other).

FIGS. 7A and 7B show correlation between significant proteomic changes in animals of different genotypes. FIG. 7A shows protein levels of Tpp1^{-/-}; Cln3^{+/+} compared to those of Tpp1^{-/-}; Cln3^{-/-}; goodness of fit, R=0.7901. NNT is censored (see Examples). FIG. 7B shows protein levels of Tpp^{+/+}; Cln3^{-/-} compared to those of Tpp1^{+/-}; Cln3^{-/-}; goodness of fit, R=0.5752. Compared to wild type, proteins shown are significantly altered in at least one model (FDR 1%), have a magnitude of change of ≥1.5 fold in at least one model, and have a consistent direction of change in both models. Names of select proteins of interest are shown in red (lysosomal) or black (other). Filled symbols indicate proteins that are significantly altered in both genotypes being compared, open symbols indicate proteins that are significantly altered in one of the two genotypes being compared.

FIG. 8 shows TPP1 activity in mutant mice. Activities measured in each genotype were compared to wild type using Dunnett's multiple comparison test. ns, not significant; **, p<0.01; ***, P<0.001.

FIGS. 9A, 9B, 9C, 9D, and 9E show TPP1 expression in mice expressing Tg^{TPP1+}. FIG. 9A shows that the transgene Tg^{TPP1+} is integrated into ROSA26 and drives mouse TPP1 expression from the chicken-actin (CAG) promoter using a synthetic intron. FIG. 9B shows expression of TPP1 activity driven by Tg^{TPP1+}. FIG. 9C shows effect of Tg^{TPP1+} on survival. The hemizygous mouse strain containing this transgene is designated as R26Tg^{TPP1+/0} a mouse lacking the TPP1 transgene is designated R26Tg^{0/0}. In double-label immunofluorescence images, TPP1 and the neuronal marker NeuN (FIG. 9D) or the microglial marker Iba1 (FIG. 9E), as well as Hoescht stained nuclei, are indicated.

FIG. 10 shows SCMAS staining in liver.

FIG. 11 shows SCMAS staining in spleen FIG. 12 shows SCMAS staining in brain.

FIG. 13 shows quantitation of SCMAS storage. Area of image occluded by SCMAS-containing inclusions was quantified in a genotype blinded analysis using ImageJ. Data were analyzed using unpaired one-way ANOVA with the mean of each sample compared to the mean of all other samples using the Tukey test to correct for multiple comparisons. Note that while all possible comparisons were tested, we only show the pairwise comparison bar for Tg-Cln3^{-/-} and Tg+Cln3^{-/-}. ****.

### DETAILED DESCRIPTION OF THE INVENTION

This disclosure provides methods of treating lysosomal storage diseases by increasing the level or activity of tripeptidyl peptidase 1 (TPP1). This disclosure also provides novel animal models for use in studying lysosomal storage diseases. In particular, the animal model having a Tpp1^{+/-}; Cln3^{-/-} double knockout has truncated survival compared to the wild type animal, making it useful in developing therapies for lysosomal storage diseases, such as juvenile neuronal ceroid lipofuscinosis (JNCL), using survival as an endpoint.

### Methods of Treating Lysosomal Storage Diseases

In one aspect, this disclosure provides a method of treating a subject having a disease or disorder characterized by accumulation of SCMAS in the lysosomes of affected cells (e.g., neuronal cells). In some embodiments, the method comprises administering to the subject a therapeutically effective amount of an agent that increases a level or activity of TPP1, to reduce or eliminate symptoms caused by the disease or disorder.

In some embodiments, the method comprises selecting a subject having an elevated level of accumulation of SCMAS in the lysosomes of affected cells compared to a reference level.

In some embodiments, the method comprises: (a) obtaining a sample containing the affected cells; (b) performing an assay on the sample and determining the level of accumulation of SCMAS in the lysosomes of the affected cells; (c) identifying the subject as likely to benefit from treatment with an agent that increases a level or activity of TPP1 if the subject has an elevated level of accumulation of SCMAS in the lysosomes of affected cells compared to a reference level; and (d) administering to the subject a therapeutically effective amount of the agent to reduce or eliminate symptoms caused by the disease or disorder.

In some embodiments, the disease or disorder is characterized by accumulation of one or more storage products (e.g., SCMAS) in the lysosomes of the affected cells, such as neurons. One mode of determining the disorder is finding that the lysosomes have accumulated storage material, which can be done by known methods such as microscopy or immunofluorescence. An example of a storage material that would be detected in lysosomes is SCMAS. In some embodiments, treatment with TPP1 protein will reduce or eliminate mitochondrial ATP synthase, in particular SCMAS in the lysosomes of the affected cells, such as neurons. Detecting elimination of storage material such as mitochondria SCMAS in the lysosomes of affected cells can be done by known methods as described above.

In some embodiments, the disease or disorder is selected from Late-infantile neuronal ceroid lipofuscinosis (CLN2) caused by mutations in the Tpp1 gene, Juvenile neuronal ceroid lipofuscinosis (CLN3) caused by mutations in the Cln3 gene, Variant late infantile neuronal ceroid lipofuscinosis type 5 (CLN5) disease caused by mutations in the Cln5 gene, Variant late infantile neuronal ceroid lipofuscinosis type 6 (CLN6) disease caused by mutations in the Cln6 gene, Neuronal ceroid lipofuscinosis type 7 (CLN7) disease caused by mutations in the MFSD8 gene, Northern epilepsy neuronal ceroid lipofuscinosis type 8 (CLN8) disease caused by mutations in the Cln8 gene, Congenital neuronal ceroid lipofuscinosis type 10 (CLN10) disease caused by mutations in the CTSD gene, Late-onset Neuronal ceroid lipofuscinosis (CLN12) and Kufor-Rakeb syndrome caused my mutations in the gene ATP13A2, Sanfilippo D syndrome (mucopolysaccharidosis type HID) caused by mutations in the GNS gene, and Osteopetrosis autosomal recessive 4 (OPTB4) caused by mutations in the CLCN7 gene.

In some embodiments, the disease or disorder is characterized by a deficiency in a function of a CLN3 protein. An example of such a disorder is JNCL.

In some embodiments, the affected cells may belong to any cell or tissue type, such as neurons. In some embodiments, the affected cells are neuronal cells. In some embodiments, the affected cells are in a tissue or organ, such as liver, spleen, or brain.

The level or activity of TPP1 may be measured by determining or estimating a protein level or mRNA level. Methods for determining or estimating a protein level or mRNA level are well known in the art. Such methods may include enzyme activity assays, microscopy, immunofluorescence, and nucleic acid hybridization (e.g., using proteins and nucleic acids described in U.S. Ser. No. 08/931,608 and Sleat et al. (1997)). For example, the protein level (e.g., protein expression level) of TPP1 can be determined by SDS-PAGE, Western blot, or an immunoassay (e.g., immunoblotting assay, immunoprecipitation assay). The mRNA level may be determined by RT-PCR.

In some embodiments, the reference level may be obtained from the subject prior to the administration of the agent for increasing a level or activity of TPP1 or a composition thereof. In some embodiments, the reference level may be obtained from a control subject or a group of individuals who do not have a disease or disorder or have not been diagnosed with a disease or disorder. In some embodiments, the reference level is obtained based on average levels of the level or activity of TPP1, for example, of a population not suffering from a disease or disorder. In some embodiments, the reference level is obtained based on a median or median level of a set of individuals in which patients with a disease or disorder are included.

In some embodiments, the agent reduces the level of accumulation of the SCMAS in the lysosomes of the affected cells. In some embodiments, the affected cells are in a tissue or organ, such as liver, spleen, or brain. In some embodiments, the agent reduces the level of accumulation of the SCMAS in the lysosomes of liver, spleen, and/or brain.

In some embodiments, the agent comprises a protein, a peptide, a peptidomimetic, a nucleic acid, or a small molecule.

In some embodiments, the agent comprises a recombinant human TPP1 protein. In some embodiments, the TPP1 protein is an inactive proenzyme. In some embodiments, the TPP1 protein is mannose-6-phosphorylated.

In some embodiments, the agent is a TPP1 protein or a variant thereof. In some embodiments, the TPP1 protein comprises an amino acid sequence having at least 75% (e.g., 75%, 80%, 85%, 90%, 95%, 99%) sequence identity with the amino acid sequence of SEQ ID NO: 1, or comprises the amino acid sequence of SEQ ID NO: 1.

In some embodiments, the TPP1 protein is encoded by a nucleotide sequence having at least 75% (e.g., 75%, 80%, 85%, 90%, 95%, 99%) sequence identity with the nucleotide sequence of SEQ ID NO: 2, or is encoded by the nucleotide sequence of SEQ ID NO: 2.

SEQ ID NO: 1
MGLQACLLGLFALILSGKCSYSPEPDQRRTLPPGWVSLGRADPEE
ELSLTFALRQQNVERLSELVQAVSDPSSPQYGKYLTLENVADLVR
PSPLTLHTVQKWLLAAGAQKCHSVITQDFLTCWLSIRQAELLLPG
AEFHHYVGGPTETHVVRSPHPYQLPQALAPHVDFVGGLHRFPPTS
SLRQRPEPQVTGTVGLHLGVTPSVIRKRYNLTSQDVGSGTSNNSQ
ACAQFLEQYFHDSDLAQFMRLFGGNFAHQASVARVVGQQGRGRAG
IEASLDVQYLMSAGANISTWVYSSPGRHEGQEPFLQWLMLLSNES
ALPHVHTVSYGDDEDSLSSAYIQRVNTELMKAAARGLTLLFASGD
SGAGCWSVSGRHQFRPTFPASSPYVTTVGGTS FQEPFLITNEIV
DYISGGGFSNVFPRPSYQEEAVTKELSSSPHLPPSSYENASGRAY
PDVAALSDGYWVVSNRVPIPWVSGTSASTPVFGGILSLINEHRIL
SGRPPLGELNPRLYQQHGAGLFDVTRGCHESCLDEEVEGQGFCSG
PGWDPVTGWGTPNFPALLKTLLNP
SEQ ID NO: 2
ATGGGACTCCAAGCCTGCCTCCTAGGGCTCTTTGCCCTCATCCTC
TCTGGCAAATGCAGTTACAGCCCGGAGCCCGACCAGCGGAGGACG
CTGCCCCCAGGCTGGGTGTCCCTGGGCCGTGCGGACCCTGAGGAA
GAGCTGAGTCTCACCTTTGCCCTGAGACAGCAGAATGTGGAAAGA
CTCTCGGAGCTGGTGCAGGCTGTGTCGGATCCCAGCTCTCCTCAA
TACGGAAAATACCTGACCCTAGAGAATGTGGCTGATCTGGTGAGG
CCATCCCCACTGACCCTCCACACGGTGCAAAAATGGCTCTTGGCA
GCCGGAGCCCAGAAGTGCCATTCTGTGATCACACAGGACTTTCTG
ACTTGCTGGCTGAGCATCCGACAAGCAGAGCTGCTGCTCCCTGGG
GCTGAGTTTCATCACTATGTGGGAGGACCTACGGAAACCCATGTT
GTAAGGTCCCCACATCCCTACCAGCTTCCACAGGCCTTGGCCCCC
CATGTGGACTTTGTGGGGGGACTGCACCGTTTTCCCCCAACATCA
TCCCTGAGGCAACGTCCTGAGCCGCAGGTGACAGGGACTGTAGGC
CTGCATCTGGGGGTAACCCCCTCTGTGATCCGTAAGCGATACAAC
TTGACCTCACAAGACGTGGGCTCTGGCACCAGCAATAACAGCCAA
GCCTGTGCCCAGTTCCTGGAGCAGTATTTCCATGACTCAGACCTG
GCTCAGTTCATGCGCCTCTTCGGTGGCAACTTTGCACATCAGGCA
TCAGTAGCCCGTGTGGTTGGACAACAGGGCCGGGGCCGGGCCGGG
ATTGAGGCCAGTCTAGATGTGCAGTACCTGATGAGTGCTGGTGCC
AACATCTCCACCTGGGTCTACAGTAGCCCTGGCCGGCATGAGGGA
CAGGAGCCCTTCCTGCAGTGGCTCATGCTGCTCAGTAATGAGTCA
GCCCTGCCACATGTGCATACTGTGAGCTATGGAGATGATGAGGAC
TCCCTCAGCAGCGCCTACATCCAGCGGGTCAACACTGAGCTCATG
AAGGCTGCCGCTCGGGGTCTCACCCTGCTCTTCGCCTCAGGTGAC
AGTGGGGCCGGGTGTTGGTCTGTCTCTGGAAGACACCAGTTCCGC
CCTACCTTCCCTGCCTCCAGCCCCTATGTCACCACAGTGGGAGGC
ACATCCTTCCAGGAACCTTTCCTCATCACAAATGAAATTGTTGAC
TATATCAGTGGTGGTGGCTTCAGCAATGTGTTCCCACGGCCTTCA
TACCAGGAGGAAGCTGTAACGAAGTTCCTGAGCTCTAGCCCCCAC
CTGCCACCATCCAGTTACTTCAATGCCAGTGGCCGTGCCTACCCA
GATGTGGCTGCACTTTCTGATGGCTACTGGGTGGTCAGCAACAGA
GTGCCCATTCCATGGGTGTCCGGAACCTCGGCCTCTACTCCAGTG
TTTGGGGGGATCCTATCCTTGATCAATGAGCACAGGATCCTTAGT
GGCCGCCCCCCTCTTGGCTTTCTCAACCCAAGGCTCTACCAGCAG
CATGGGGCAGGACTCTTTGATGTAACCCGTGGCTGCCATGAGTCC
TGTCTGGATGAAGAGGTAGAGGGCCAGGGTTTCTGCTCTGGTCCT
GGCTGGGATCCTGTAACAGGCTGGGGAACACCCAACTTCCCAGCT
TTGCTGAAGACTCTACTCAACCCCTGA

As used herein, the term “variant” refers to a first molecule that is related to a second molecule (also termed a “parent” molecule). The variant molecule can be derived from, isolated from, based on or homologous to the parent molecule. For example, the mutant forms of TPP1, including the TPP1 mutant with a cysteine substitution, are variants of the wild type TPP1. The term variant can be used to describe either polynucleotides or polypeptides.

As applied to proteins, a variant polypeptide can have an entire amino acid sequence identity with the original parent polypeptide or can have less than 100% amino acid identity with the parent protein. For example, a variant of an amino acid sequence can be a second amino acid sequence that is at least 50%, 60%, 70%, 80%, 90%, 95%, 98%, 99% or more identical in amino acid sequence compared to the original amino acid sequence. Polypeptide variants include polypeptides comprising the entire parent polypeptide, and further comprising additional fused amino acid sequences. Polypeptide variants also include polypeptides that are portions or subsequences of the parent polypeptide. For example, unique subsequences (e.g., as determined by standard sequence comparison and alignment techniques) of the polypeptides disclosed herein are also encompassed by the invention.

In another aspect, polypeptide variants include polypeptides that contain minor, trivial, or inconsequential changes to the parent amino acid sequence. For example, minor, trivial, or inconsequential changes include amino acid changes (including substitutions, deletions, and insertions) that have little or no impact on the biological activity of the polypeptide, and yield functionally identical polypeptides, including additions of non-functional peptide sequence. In other aspects, the variant polypeptides of the invention change the biological activity of the parent molecule. One of skill will appreciate that many variants of the disclosed polypeptides are encompassed by the invention.

In some aspects, polynucleotide or polypeptide variants of the invention can include variant molecules that alter, add or delete a small percentage of the nucleotide or amino acid positions, for example, typically less than about 10%, less than about 5%, less than 4%, less than 2% or less than 1%.

A “functional variant” of a protein as used herein refers to a variant of such protein that retains at least partially the activity of that protein. Functional variants may include mutants (which may be insertion, deletion, or replacement mutants), including polymorphs, etc. Also included within functional variants are fusion products of such protein with another, usually unrelated, nucleic acid, protein, polypeptide, or peptide. Functional variants may be naturally occurring or may be man-made.

In some embodiments, the TPP1 variant may include one or more conservative modifications. The TPP1 variant with one or more conservative modifications may retain the desired functional properties, which can be tested using the functional assays known in the art. As used herein, the term “conservative sequence modifications” refers to amino acid modifications that do not significantly affect or alter the binding characteristics of the protein containing the amino acid sequence. Such conservative modifications include amino acid substitutions, additions, and deletions. Modifications can be introduced by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include: amino acids with basic side chains (e.g., lysine, arginine, histidine); acidic side chains (e.g., aspartic acid, glutamic acid); uncharged polar side chains (e.g., glycine, asparagine, glutamine, serine, threonine, tyrosine, cysteine, tryptophan); nonpolar side chains (e.g., alanine, valine, leucine, isoleucine, proline, phenylalanine, methionine); beta-branched side chains (e.g., threonine, valine, isoleucine); and aromatic side chains (e.g., tyrosine, phenylalanine, tryptophan, histidine) includes one or more conservative modifications. The Cas protein with one or more conservative modifications may retain the desired functional properties, which can be tested using the functional assays known in the art. As used herein, the term “conservative sequence modifications” refers to amino acid modifications that do not significantly affect or alter the binding characteristics of the protein containing the amino acid sequence. Such conservative modifications include amino acid substitutions, additions, and deletions. Modifications can be introduced by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include: amino acids with basic side chains (e.g., lysine, arginine, histidine); acidic side chains (e.g., aspartic acid, glutamic acid); uncharged polar side chains (e.g., glycine, asparagine, glutamine, serine, threonine, tyrosine, cysteine, tryptophan); nonpolar side chains (e.g., alanine, valine, leucine, isoleucine, proline, phenylalanine, methionine); beta-branched side chains (e.g., threonine, valine, isoleucine); and aromatic side chains (e.g., tyrosine, phenylalanine, tryptophan, histidine).

As used herein, the percent homology between two amino acid sequences is equivalent to the percent identity between the two sequences. The percent identity between the two sequences is a function of the number of identical positions shared by the sequences (i.e., % homology=#of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two amino acid sequences can be determined using the algorithm of E. Meyers and W. Miller (Comput. Appl. Biosci., 4:11-17 (1988)), which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. 48:444-453 (1970)) algorithm, which has been incorporated into the GAP program in the GCG software package (available at www.gcg.com), using either a Blossum62 matrix or a PAM250 matrix, and a gap weight of 16, 14, 12, 10, 8, 6, or 4 and a length weight of 1, 2, 3, 4, 5, or 6.

Additionally or alternatively, the protein sequences of the present invention can further be used as a “query sequence” to perform a search against public databases to, for example, identify related sequences. Such searches can be performed using the XBLAST program (version 2.0) of Altschul, et al. (1990) J. Mol. Biol. 215:403-10. BLAST protein searches can be performed with the XBLAST program, score=50, wordlength=3 to obtain amino acid sequences homologous to the antibody molecules of the invention. To obtain gapped alignments for comparison purposes, Gapped BLAST can be utilized as described in Altschul et al. (1997) Nucleic Acids Res. 25(17):3389-3402. When utilizing BLAST and Gapped BLAST programs, the default parameters of the respective programs (e.g., XBLAST and NBLAST) can be used. (See www.ncbi.nlm.nih.gov).

In some embodiments, the TPP1 variant can be conjugated or linked to a detectable tag or a detectable marker (e.g., a radionuclide, a fluorescent dye). In some embodiments, the detectable tag can be an affinity tag. The term “affinity tag” as used herein relates to a moiety attached to a polypeptide, which allows the polypeptide to be purified from a biochemical mixture. Affinity tags can consist of amino acid sequences or can include amino acid sequences to which chemical groups are attached by post-translational modifications. Non-limiting examples of affinity tags include His-tag, CBP-tag (CBP: calmodulin-binding protein), CYD-tag (CYD: covalent yet dissociable NorpD peptide), Strep-tag, StrepII-tag, FLAG-tag, HPC-tag (HPC: heavy chain of protein C), GST-tag (GST: glutathione S transferase), Avi-tag, biotinylated tag, Myc-tag, 3×FLAG tag, a SUMO tag, and MBP-tag (MBP: maltose-binding protein). Further examples of affinity tags can be found in Kimple et al., Curr Protoc Protein Sci. 2013 Sep. 24; 73: Unit 9.9.

In some embodiments, the detectable tag can be conjugated or linked to the N- and/or C-terminus of the TPP1 variant. The detectable tag and the affinity tag may also be separated by one or more amino acids. In some embodiments, the detectable tag can be conjugated or linked to the TPP1 variant via a cleavable element. In the context of the present invention, the term “cleavable element” relates to peptide sequences that are susceptible to cleavage by chemical agents or enzyme means, such as proteases. Proteases may be sequence-specific (e.g., thrombin) or may have limited sequence specificity (e.g., trypsin). Cleavable elements I and II may also be included in the amino acid sequence of a detection tag or polypeptide, particularly where the last amino acid of the detection tag or polypeptide is K or R.

As used herein, the term “conjugate” or “conjugation” or “linked” as used herein refers to the attachment of two or more entities to form one entity. A conjugate encompasses both peptide-small molecule conjugates as well as peptide-protein/peptide conjugates.

The term “fusion polypeptide” or “fusion protein” means a protein created by joining two or more polypeptide sequences together. The fusion polypeptides encompassed in this invention include translation products of a chimeric gene construct that joins the nucleic acid sequences encoding a first polypeptide with the nucleic acid sequence encoding a second polypeptide to form a single open reading frame. In other words, a “fusion polypeptide” or “fusion protein” is a recombinant protein of two or more proteins that are joined by a peptide bond or via several peptides. The fusion protein may also comprise a peptide linker between the two domains.

In some embodiments, the agent comprises a fusion protein comprising a TPP1 or a variant thereof.

In some embodiments, the agent comprises a nucleic acid (e.g., DNA, RNA) having a polynucleotide sequence encoding the TPP1 protein or the variant thereof. In some embodiments, the polynucleotide sequence is RNA. In some embodiments, the agent comprises a vector having a polynucleotide sequence encoding the TPP1 protein or the variant thereof.

The term “vector” or “expression vector” is synonymous with “expression construct” and refers to a DNA molecule that is used to introduce and direct the expression of a specific gene to which it is operably associated in a target cell. The term includes the vector as a self-replicating nucleic acid structure as well as the vector incorporated into the genome of a host cell into which it has been introduced. The expression vector of the present invention comprises an expression cassette. Expression vectors allow transcription of large amounts of stable mRNA. Once the expression vector is inside the target cell, the ribonucleic acid molecule or protein that is encoded by the gene is produced by the cellular transcription and/or translation machinery. In one embodiment, the expression vector of the invention comprises an expression cassette that comprises polynucleotide sequences that encode TPP1 or a variant thereof.

The vectors may comprise a polynucleotide that encodes an RNA (e.g., RNAi, ribozymes, miRNA, siRNA) that, when transcribed from the polynucleotides of the vector, will result in the accumulation of chimeric proteins on the plasma membranes of target cells. Vectors that may be used include, without limitation, lentiviral, HSV, and adenoviral vectors. Lentiviruses include, but are not limited to, HIV-1, HIV-2, SIV, FIV, and EIAV. Lentiviruses may be pseudotyped with the envelope proteins of other viruses, including, but not limited to VSV, rabies, Mo-MLV, baculovirus, and Ebola. Such vectors may be prepared using standard methods in the art.

In some embodiments, the vector is a recombinant AAV vector. AAV vectors are DNA viruses of relatively small size that can integrate, in a stable and site-specific manner, into the genome of the cells that they infect. They are able to infect a wide spectrum of cells without inducing any effects on cellular growth, morphology or differentiation, and they do not appear to be involved in human pathologies. The AAV genome has been cloned, sequenced, and characterized. It encompasses approximately 4700 bases and contains an inverted terminal repeat (ITR) region of approximately 145 bases at each end, which serves as an origin of replication for the virus. The remainder of the genome is divided into two essential regions that carry the encapsidation functions: the left-hand part of the genome, which contains the rep gene involved in viral replication and expression of the viral genes; and the right-hand part of the genome, which contains the cap gene encoding the capsid proteins of the virus.

The application of AAV, for example, as a vector for gene therapy, has been rapidly developed in recent years. Wild type AAV can infect, with a comparatively high titer, dividing or non-dividing cells, or tissues of a mammal, including human, and also can integrate into human cells at a specific site (on the long arm of chromosome 19) (Kotin, R. M., et al., Proc. Natl. Acad. Sci. USA 87: 2211-2215, 1990) (Samulski, R. J, et al., EMBO J. 10: 3941-3950, 1991 the disclosures of which are hereby incorporated by reference herein in their entireties). AAV vector without the rep and cap genes loses specificity of site-specific integration, but may still mediate long-term stable expression of exogenous genes. AAV vector exists in cells in two forms, wherein one is episomic outside of the chromosome; another is integrated into the chromosome, with the former as the major form. Moreover, AAV has not hitherto been found to be associated with any human disease, nor any change of biological characteristics arising from the integration has been observed. There are sixteen serotypes of AAV reported in the literature, respectively named AAV1, AAV2, AAV3, AAV4, AAV5, AAV6, AAV7, AAV8, AAV9, AAV10, AAV 11, AAV12, AAV13, AAV14, AAV15, AAV16, and recombinant variants thereof, wherein AAV5 is originally isolated from humans (Bantel-Schaal, and H. zur Hausen. 1984. Virology 134: 52-63), while AAV1-4 and AAV6 are all found in the study of adenovirus (Ursula Bantel-Schaal, Hajo Delius and Harald zur Hausen. J. Virol. 1999, 73: 939-947).

AAV vectors may be prepared using standard methods in the art. Adeno-associated viruses of any serotype are suitable (See, e.g., Blacklow, pp. 165-174 of “Parvoviruses and Human Disease” J. R. Pattison, ed. (1988); Rose, Comprehensive Virology 3:1, 1974; P. Tattersall “The Evolution of Parvovirus Taxonomy” In Parvoviruses (J R Kerr, S F Cotmore. M E Bloom, R M Linden, C R Parrish, Eds.) p 5-14, Hudder Arnold, London, U K (2006); and D E Bowles, J E Rabinowitz, R J Samulski “The Genus Dependovirus” (J R Kerr, S F Cotmore. M E Bloom, R M Linden, C R Parrish, Eds.) p 15-23, Hudder Arnold, London, UK (2006), the disclosures of which are hereby incorporated by reference herein in their entireties). Methods for purifying vectors may be found in, for example, U.S. Pat. Nos. 6,566,118, 6,989,264, and 6,995,006 and WO/1999/011764 titled “Methods for Generating High Titer Helper-free Preparation of Recombinant AAV Vectors,” the disclosures of which are herein incorporated by reference in their entireties. Preparation of hybrid vectors is described in, for example, PCT Application No. PCT/US2005/027091, the disclosure of which is herein incorporated by reference in its entirety. The use of vectors derived from the AAVs for transferring genes in vitro and in vivo has been described (See, e.g., International Patent Application Publication Nos: 91/18088 and WO 93/09239; U.S. Pat. Nos. 4,797,368, 6,596,535, and 5,139,941; and European Patent No: 0488528, all of which are herein incorporated by reference in their entireties). These publications describe various AAV-derived constructs in which the rep and/or cap genes are deleted and replaced by a gene of interest and the use of these constructs for transferring the gene of interest in vitro (into cultured cells) or in vivo (directly into an organism). The replication-defective recombinant AAVs can be prepared by co-transfecting a plasmid containing the nucleic acid sequence of interest flanked by two AAV inverted terminal repeat (ITR) regions and a plasmid carrying the AAV encapsidation genes (rep and cap genes) into a cell line that is infected with a human helper virus (for example an adenovirus). The AAV recombinants that are produced are then purified by standard techniques.

In some embodiments, the vector(s) can be encapsidated into a virus particle (e.g., AAV virus particle including, but not limited to, AAV1, AAV2, AAV3, AAV4, AAV5, AAV6, AAV7, AAV8, AAV9, AAV10, AAV11, AAV12, AAV13, AAV14, AAV15, and AAV16). Accordingly, also provided is a recombinant virus particle (recombinant because it contains a recombinant polynucleotide) comprising any of the vectors described herein. Methods of producing such particles are known in the art and are described in U.S. Pat. No. 6,596,535.

In some embodiments, the viral vector comprises an AAV vector, lentiviral vector, adenoviral vector, or a non-viral plasmid vector. In some embodiments, the adeno-associated viral vector is selected from AAV1, AAV2, AAV3, AAV4, AAV5, AAV6, AAV7, AAV8, AAV9, AAV10, AAV11, AAV 12, AAV13, AAV rh74, and recombinant subtypes thereof.

In some embodiments, vectors can be derived from retroviruses, including avian reticuloendotheliosis virus (duck infectious anemia virus, spleen necrosis virus, Twiehaus-strain reticuloendotheliosis virus, C-type retrovirus, reticuloendotheliosis virus Hungary-2 (REV-H-2)), and feline leukemia virus (FeLV)). Retroviral genomes have been modified for use as a vector (Cone & Mulligan, Proc. Natl. Acad. Sci., USA, 81:6349-6353, (1984)). Non-limiting examples of retroviruses include lentiviruses, such as human immunodeficiency viruses (HIV-1 and HIV-2), feline immunodeficiency virus (FIV), simian immunodeficiency virus (SIV), Maedi/Visna virus, caprine arthritis/encephalitis virus, equine infectious anaemia virus (EIAV), and bovine immunodeficiency virus (BIV); avian type C retroviruses, such as the avian leukosis virus (ALV); HTLV-BLV retroviruses, such as bovine leukaemia virus (BLV), human T cell lymphotropic virus (HTLV), and simian T cell lymphotropic virus; mammalian type B retroviruses, such as the mouse mammary tumor virus (MMTV); mammalian type C retroviruses, such as the murine leukaemia virus (MLV), feline sarcoma virus (FeSV), murine sarcoma virus, Gibbon ape leukemia virus, guinea pig type C virus, porcine type C virus, wooly monkey sarcoma virus, and viper retrovirus; spumavirus (foamy virus group), such as human spumavirus (HSRV), feline synctium-forming virus (FeSFV), human foamy virus, simian foamy virus, and bovine syncytial virus; and type D retroviruses, such as Mason-Pfizer monkey virus (MPMV), squirrel monkey retrovirus, and langur monkey virus.

In some embodiments, the vector comprises a retroviral vector or a lentiviral vector. In some embodiments, lentiviral and retroviral vectors may be packaged using their native envelope proteins or may be modified to be encapsulated with heterologous envelope proteins. Examples of envelope proteins include, but are not limited to, an amphotropic envelope, an ecotropic envelope, or a xenotropic envelope, or may be an envelope including amphotropic and ecotropic portions. The protein also may be that of any of the above-mentioned retroviruses and lentiviruses. Alternatively, the env proteins may be modified, synthetic or chimeric env constructs, or may be obtained from non-retro viruses, such as vesicular stomatitis virus and HVJ virus. Specific non-limiting examples include the envelope of Moloney Murine Leukemia Virus (MMLV), Rous Sarcoma Virus, Baculovirus, Jaagsiekte Sheep Retrovirus (JSRV) envelope protein, and the feline endogenous virus RD114; gibbon ape leukemia virus (GALV) envelope; baboon endogenous virus (BaEV) envelope; simian sarcoma-associated virus (SSAV) envelope; amphotropic murine leukemia virus (MLV-A) envelope; human immunodeficiency virus envelope; avian leukosis virus envelope; the endogenous xenotropic NZB viral envelopes; and envelopes of the paramyxoviridiae family such as, but not limited to, the HVJ virus envelope.

Once the expression vector or DNA sequence containing the constructs has been prepared for expression, the expression vectors can be transfected or introduced into an appropriate host cell. Various techniques may be employed to achieve this, such as, for example, protoplast fusion, calcium phosphate precipitation, electroporation, retroviral transduction, viral transfection, gene gun, lipid-based transfection, or other conventional techniques. Methods and conditions for culturing the resulting transfected cells and for recovering the expressed polypeptides are known to those skilled in the art and may be varied or optimized depending upon the specific expression vector and mammalian host cell employed, based upon the present description.

In some embodiments, the agent is a TPP1 agonist. The term “agonist” refers to a compound that causes agonism of the TPP1 pathway and causes a response in a cell. The TPP1 agonist mimics the action of an endogenous ligand (TPP1), resulting in a physiological response similar to that provided by the endogenous ligand. The term includes agents (e.g., a TPP1 variant/fragment, a fusion protein comprising TPP1 that, upon administration to a subject in need thereof, cause an upregulation and/or an increase in the activity of TPP1-mediated signaling pathways. In some embodiments, the term includes an agent that leads to a reduction in the number or activity of T cells upon administration to a subject.

The TPP1 protein may be in an inactive proenzyme (or prodrug) form, or in the shorter active form. Either of these may be naturally isolated, or recombinant. When produced, for example, in Chinese hamster ovary (CHO) cells, TPP1 protein in its proenzyme form is obtained. This form converts to the active form following acidification. Therefore, the proenzyme is a highly suitable prodrug that remains inactive until delivered to lysosomes, whose acidic environment will activate it. Obtaining TPP1 protein in any of these forms is described in detail below and in U.S. Ser. No. 08/931,608 and Sleat et al. (1997). Briefly, TPP1 protein can be isolated using known methods from human brain samples by purifying mannose-6-phosphate containing glycoproteins from normal, JNCL brain samples, or LINCL brain samples and isolating the protein band present in the normal but not in the JNCL or LINCL specimens. Once the protein is obtained, the corresponding gene and cDNA are also isolated using known methods. Recombinant protein is then produced from the cDNA using known methods. The TPP1 protein in any of the above forms may or may not be mannose-6-phosphorylated.

In some embodiments, the agent may include an agonist of peroxisome proliferator-activated receptor α (PPARα) or a stereoisomer thereof, a derivative thereof, an analog thereof, a prodrug thereof, or a pharmaceutically acceptable salt thereof.

As used herein, “PPARα agonist” refers to a compound or composition which when combined with PPARα directly or indirectly (preferably binding directly to PPARα) stimulates or increases an in vivo or in vitro reaction typical for the receptor, e.g., transcriptional regulation activity, as measured by an assay known to one skilled in the art, including, but not limited to, the “co-transfection” or “cistrans” assays described or disclosed in U.S. Pat. Nos. 4,981,784, 5,071,773, 5,298,429, 5,506,102, WO89/05355, WO91/06677, WO92/05447, WO93/11235, WO93/23431, WO94/23068, WO95/18380, CA 2,034,220, and Lehmann, et al., J. Biol. Chem. 270:12953-12956 (1995), which are incorporated by reference herein. PPARα agonists may also be identified according to an assay described in U.S. Pat. No. 6,008,239.

In some embodiments, PPARα agonists may include a fibrate compound including, but not limited to, gemfibrozil, fenofibrate, bezafibrate, clofibrate, ciprofibrate, and analogues, derivatives and pharmaceutically acceptable salts thereof. PPARα compounds disclosed in Tontonez et al., Cell 79:1147-1156 (1994), Lehmann et al., J. Biol. Chem. 270(22):1-4, 1995, Amri et al., J. Lipid Res. 32:14491456 (1991), Kliewer et al., Proc. Natl. Acad. Sci. USA 94:4318-4323 (1997), Amri et al., J. Lipid Res. 32:1457-1463, (1991) and Grimaldi et al., Proc. Natl. Acad. Sci. USA 89:10930-10934 (1992) are incorporated by reference herein. PPARα agonist compounds described in U.S. Pat. No. 6,008,239, WO 97/27847, WO 97/27857, WO 97/28115, WO 97/28137 and WO 97/28149 are further incorporated by reference herein. Certain fibrate compounds as described in WO92/10468 and WO01/80852 are also incorporated by reference herein.

“Isomers” are different compounds that have the same molecular formula. “Stereoisomers” are isomers that differ only in the way the atoms are arranged in space, i.e., having a different stereochemical configuration. “Enantiomers” are a pair of stereoisomers that are non-superimposable mirror images of each other. A 1:1 mixture of a pair of enantiomers is a “racemic” mixture. The term “(±)” is used to designate a racemic mixture where appropriate. “Diastereoisomers” are stereoisomers that have at least two asymmetric atoms, but which are not mirror-images of each other. The absolute stereochemistry is specified according to the Cahn-Ingold-Prelog R-S system. When a compound is a pure enantiomer, the stereochemistry at each chiral carbon can be specified by either (R) or (S). Resolved compounds whose absolute configuration is unknown can be designated (+) or (−) depending on the direction (dextro- or levorotatory) in which they rotate plane polarized light at the wavelength of the sodium D line. Certain of the compounds described herein contain one or more asymmetric centers and can thus give rise to enantiomers, diastereomers, and other stereoisomeric forms that can be defined, in terms of absolute stereochemistry, as (R) or (S). The present chemical entities, pharmaceutical compositions, and methods are meant to include all such possible isomers, including racemic mixtures, optically pure forms, and intermediate mixtures. Optically active (R)- and (S)-isomers can be prepared using chiral synthons or chiral reagents or resolved using conventional techniques. When the compounds described herein contain olefinic double bonds or other centers of geometric asymmetry, and unless specified otherwise, it is intended that the compounds include both E and Z geometric isomers.

Stereoisomers are compounds that differ only in their spatial arrangement. Enantiomers are pairs of stereoisomers whose mirror images are not superimposable, most commonly because they contain an asymmetrically substituted carbon atom that acts as a chiral center. “Enantiomer” means one of a pair of molecules that are mirror images of each other and are not superimposable. Diastereomers are stereoisomers that are not related to mirror images, most commonly because they contain two or more asymmetrically substituted carbon atoms. “R” and “S” represent the configuration of substituents around one or more chiral carbon atoms. Thus, “R*” and “S*” denote the relative configurations of substituents around one or more chiral carbon atoms. The symbol “*” in a structural formula represents the presence of a chiral carbon center.

“Racemate” or “racemic mixture” means a compound of equimolar quantities of two enantiomers, wherein such mixtures exhibit no optical activity, i.e., they do not rotate the plane of polarized light.

“Geometric isomer” means isomers that differ in the orientation of substituent atoms in relationship to a carbon-carbon double bond, to a cycloalkyl ring, or to a bridged bicyclic system. Atoms (other than H) on each side of a carbon-carbon double bond may be in an E (substituents are on opposite sides of the carbon-carbon double bond) or Z (substituents are oriented on the same side) configuration. “R,” “S,” “St,” “R*,” “E,” “Z,” “cis,” and “trans” indicate configurations relative to the core molecule.

A “derivative,” as used herein, refers to a chemical substance related structurally to another, i.e., an “original” substance, which can be referred to as a “parent” compound. A “derivative” can be made from the structurally-related parent compound in one or more steps. The phrase “closely related derivative” means a derivative whose molecular weight does not exceed the weight of the parent compound by more than 50%. The general physical and chemical properties of a closely related derivative are also similar to the parent compound. “Pharmaceutically active derivative” refers to any compound that, upon administration to the recipient, is capable of providing, directly or indirectly, the activity disclosed herein.

An “analog” refers to a small organic compound, a nucleotide, a protein, or a polypeptide that possesses similar or identical activity or function(s) as the compound, nucleotide, protein or polypeptide or compound having the desired activity of this disclosure, but need not necessarily may include a sequence or structure that is similar or identical to the sequence or structure of the preferred embodiments.

A “prodrug” refers to a compound that may be converted under physiological conditions or by solvolysis to a biologically active compound described herein. Thus, the term “prodrug” refers to a precursor of a biologically active compound that is pharmaceutically acceptable. A prodrug may be inactive when administered to a subject, but is converted in vivo to an active compound, for example, by hydrolysis. The prodrug compound often offers the advantages of solubility, tissue compatibility, or delayed release in a mammalian organism (see, e.g., Bundgaard, H., Design of Prodrugs (1985) (Elsevier, Amsterdam). The term “prodrug” also refers to any covalently bonded carriers, which release the active compound in vivo when administered to a subject. Prodrugs of an active compound, as described herein, may be prepared by modifying functional groups present in the active compound in such a way that the modifications are cleaved, either in routine manipulation or in vivo, to yield the active parent compound. Prodrugs include, for example, compounds wherein a hydroxy, amino or mercapto group is bonded to any group that, when the prodrug of the active compound is administered to a mammalian subject, cleaves to form a free hydroxy, free amino, or free mercapto group, respectively. Examples of prodrugs include, but are not limited to, acetates, formates, and benzoate derivatives of alcohol, various ester derivatives of a carboxylic acid, or acetamide, formamide, and benzamide derivatives of an amine functional group in the active compound. Various forms of prodrugs are well known in the art and are described in: (a) The Practice of Medicinal Chemistry, Camille G. Wermuth et al., Ch 31, (Academic Press, 1996); (b) Design of Prodrugs, edited by H. Bundgaard, (Elsevier, 1985); (c) A Textbook of Drug Design and Development, P. Krogsgaard-Larson and H. Bundgaard, eds. Ch 5, pgs 113-191 (Harwood Academic Publishers, 1991); and (d) Hydrolysis in Drug and Prodrug Metabolism, Bernard Testa and Joachim M. Mayer, (Wiley-VCH, 2003).

In some embodiments, the agent for increasing a level or activity of TPP1 may be provided as a composition, e.g., a pharmaceutical composition. The composition may include one or a combination of the TPP1 protein or variants thereof, formulated together with a pharmaceutically acceptable carrier. In some embodiments, such compositions may include one or a combination of (e.g., two or more different) TPP1 variants. For example, the composition can include a combination of the TPP1 variants having different genetic modifications.

Accordingly, also provided in this disclosure is a pharmaceutical composition for treating a disease or disorder in a subject. The pharmaceutical composition comprises: (a) an agent capable of increasing a level or activity of TPP1 in a subject and (b) optionally a pharmaceutically acceptable carrier. In some embodiments, the agent comprises a fusion protein comprising a TPP1 protein or a variant/fragment thereof or a fusion protein comprising a TPP1 protein or a variant thereof.

As used herein, the term “composition” or “pharmaceutical composition” refers to a mixture of at least one component useful within the invention with other components, such as carriers, stabilizers, diluents, dispersing agents, suspending agents, thickening agents, and/or excipients. The pharmaceutical composition facilitates administration of one or more components of the invention to an organism.

Pharmaceutical compositions or therapeutic formulations of the agent for increasing a level or activity of TPP1 (e.g., TPP1 protein or a variant thereof, TPP1 agonist) can be prepared by mixing the agent thereof having the desired degree of purity with optional physiologically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed and include buffers such as phosphate, citrate, and other organic acids; antioxidants including ascorbic acid and methionine; preservatives (e.g., octadecyldimethylbenzyl ammonium chloride; hexamethonium chloride; benzalkonium chloride, benzethonium chloride; phenol, butyl or benzyl alcohol; alkyl parabens such as methyl or propylparaben; catechol; resorcinol; cyclohexanol; 3-pentanol; and m-cresol); low molecular weight (less than about 10 residues) proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN, PLURONIC, or polyethylene glycol (PEG).

The formulation may also contain more than one active ingredient as necessary for the particular indication being treated, preferably those with complementary activities that do not adversely affect each other. For instance, the formulation may further comprise another anti-inflammation agent. Such molecules are suitably present in combination in amounts that are effective for the purpose intended.

The active ingredients may also be entrapped in microcapsule prepared, for example, by coacervation techniques or by interfacial polymerization, for example, hydroxymethyl cellulose or gelatin-microcapsule and poly-(methylmethacrylate) microcapsule, respectively, in colloidal drug delivery systems (for example, liposomes, albumin microspheres, microemulsions, nano-particles, and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the agent for increasing a level or activity of TPP1 (e.g., the TPP1 protein or a variant thereof), which matrices are in the form of shaped articles, e.g., films, or microcapsule. Examples of sustained-releasable matrices include polyesters, hydrogels (for example, poly(2-hydroxyethyl-methacrylate), or poly(vinylalcohol)), polylactides (U.S. Pat. No. 3,773,919), copolymers of L-glutamic acid and gamma-ethyl-L-glutamate, non-degradable ethylene-vinyl acetate, degradable lactic acid-glycolic acid copolymers such as the LUPRON DEPOT (injectable microspheres composed of lactic acid-glycolic acid copolymer and leuprolide acetate), and poly-d(−)-3-hydroxybutyric acid (PHB). While polymers such as ethylene-vinyl acetate and lactic acid-glycolic acid enable release of molecules for over 100 days, certain hydrogels release proteins for shorter time periods. When encapsulated, the agent remains in the body for a long time. It may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thiol-disulfide interchange, stabilization may be achieved by modifying sulfhydryl residues, lyophilizing from acidic solutions, controlling moisture content, using appropriate additives, and developing specific polymer matrix compositions.

The formulations to be used for in vivo administration must be sterile, which can be readily accomplished by filtration through sterile filtration membranes. Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization), which yield a powder of the active ingredient plus any additional desired ingredient from a previously sterile-filtered solution thereof.

The amount of active ingredient which can be combined with a carrier material to produce a single dosage form will vary depending on the subject being treated and the particular mode of administration. The amount of active ingredient which can be combined with a carrier material to produce a single dosage form will generally be that amount of the composition which produces a therapeutic effect. Generally, out of one hundred percent, this amount will range from about 0.01 percent to about ninety-nine percent of active ingredient, preferably from about 0.1 percent to about 70 percent, most preferably from about 1 percent to about 30 percent of active ingredient in combination with a pharmaceutically acceptable carrier.

The agent for increasing a level or activity of TPP1 (e.g., TPP1 protein or a variant thereof) can be administered as a single dose or, more commonly, can be administered on multiple occasions. Intervals between single dosages can be, for example, weekly, monthly, every three months or yearly. Intervals can also be irregular, as indicated by measuring blood levels of TPP1 protein or a variant thereof in the patient.

The agent or the pharmaceutical composition thereof can be administered via one or more routes of administration using one or more of a variety of methods known in the art. As will be appreciated by the skilled artisan, the route and/or mode of administration will vary depending upon the desired results. For example, administration for the TPP1 protein or a variant thereof may include intravenous, intramuscular, intradermal, intraperitoneal, subcutaneous, spinal or other parenteral routes of administration, for example, by injection or infusion. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural, and intrasternal injection and infusion. Alternatively, a TPP1 protein or a variant thereof can be administered via a non-parenteral route, such as a topical, epidermal or mucosal route of administration, for example, intranasally, orally, vaginally, rectally, sublingually or topically.

In some embodiments, the TPP1 protein or a composition including the TPP1 protein may be introduced parenterally, transmucosally, e.g., orally, nasally, or rectally, or transdermally. Preferably, administration is by injection, especially parenteral, e.g., via intravenous injection, and also including, but is not limited to, intra-arteriole, intramuscular, intradermal, subcutaneous. intraperitoneal, intraventricular, and intracranial administration.

In some embodiments, the agent is administered by injection. In some embodiments, the injection is intracranial. In some embodiments, the agent is delivered to lysosomes of the affected cells.

TPP1 protein or composition can be delivered in a vesicle, in particular, a liposome (see Langer, Science 249:1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid). To reduce its systemic side effects and increase cellular penetration, this may be a preferred method for introducing TPP1.

TPP1 protein or composition can be delivered in a controlled release system. For example, it may be administered using intravenous infusion, an implantable osmotic pump, a transdermal patch, liposomes, or other modes of administration. In one embodiment, a pump may be used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989)). In another embodiment, polymeric materials can be used (see Medical Applications of Controlled Release, Langer and Wise (eds.), CRC Pres., Boca Raton, Fla. (1974); Controlled Drug Bioavailability, Drug Product Design, and Performance, Smolen and Ball (eds.), Wiley, New York (1984); Ranger and Peppas, J. Macromol. Sci. Rev. Macromol. Chem. 23:61 (1983); see also Levy et al., Science 228:190 (1985); During et al., Ann. Neurol. 25:351 (1989); Howard et al., J. Neurosurg. 71:105 (1989)). In yet another embodiment, a controlled release system can be placed in proximity of the therapeutic target, thus requiring only a fraction of the systemic dose (see, e.g., Goodson, in Medical Applications of Controlled Release, supra, vol. 2, pp. 115-138 (1984)). Preferably, a controlled release device is introduced into a subject in the proximity of the site LINCL-affected tissue. Other controlled release systems are discussed in the review by Langer (Science 249:1527-1533 (1990)).

Dosage regimens are adjusted to provide the optimum desired response (e.g., a therapeutic response). For example, a single bolus may be administered, several divided doses may be administered over time, or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active ingredient calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active ingredient for the treatment of sensitivity in individuals.

An amount of TPP1 protein effective to reduce or eliminate the symptoms caused by the deficiency in TPP1 protein is readily determined by the skilled practitioner. As discussed above, alleviation (i.e., reduction or elimination) of symptoms may be determined based on the physical condition of the patient, for example, cessation of seizures or reduction in amount or intensity of seizures. Or the measurement may be made on cell samples, for example, brain neurons, by determining the amount of storage products present in lysosomes and comparing with normal control cells to confirm relief of the condition. Thus the dosage may be determined by a skilled practitioner depending on the age, size, and condition of the patient. Alternatively, the amount of TPP1 protein administered may be such that normal levels of TPP1 protein in the cell + are restored, as determined, for example, by comparison to normal cells. In a preferred treatment, the effective amount of TPP1 protein is such that the affected cells receive from about 1.0 nM to about 100 nM of TPP1 protein. The dosage used to ensure the affected cells receive from about 1.0 to 100 nM of TPP1 protein may be determined by the skilled practitioner, for example, by a biopsy after administration and analysis of treated cells by known methods to determine how much injected or oral or inhaled TPP1 is required to provide the desired cell levels. When administered with an uptake inhibitor, the uptake inhibitor should be at a concentration that would inhibit immediate clearance of the TPP1 protein near the site of administration. Such a dosage may be determined by a skilled practitioner. When the uptake inhibitor is mannose-6-phosphate, 5 mM is a preferred dosage.

In some embodiments, the TPP1 protein can be administered according to one of the following dosing schedules: (i) every four weeks for six dosages, then every three months; (ii) every three weeks; and (iii) 3 mg/kg body weight once followed by 1 mg/kg body weight every three weeks.

Alternatively, the agent can be administered as a sustained release formulation, in which case less frequent administration is required. Dosage and frequency vary depending on the half-life of the agent in the patient. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of the disease. Thereafter, the patient can be administered a prophylactic regime.

Actual dosage levels of the active ingredients in the compositions may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors, including the activity of the particular compositions of the present invention employed, the route of administration, the time of administration, the rate of excretion of the particular active ingredient being employed, the duration of the treatment, other drugs, compounds and/or materials used in combination with the particular compositions employed, the age, sex, weight, condition, general health and prior medical history of the patient being treated, and like factors well known in the medical arts.

The term “effective amount,” “effective dose,” or “effective dosage” is defined as an amount sufficient to achieve or at least partially achieve a desired effect. A “therapeutically effective amount” or “therapeutically effective dosage” of a drug or therapeutic agent is any amount of the drug that, when used alone or in combination with another therapeutic agent, promotes disease regression evidenced by a decrease in severity of disease symptoms, an increase in frequency and duration of disease symptom-free periods, or a prevention of impairment or disability due to the disease affliction. A “prophylactically effective amount” or a “prophylactically effective dosage” of a drug/agent is an amount of the drug that, when administered alone or in combination with another therapeutic agent to a subject at risk of developing a disease or of suffering a recurrence of disease, inhibits the development or recurrence of the disease. The ability of a therapeutic or prophylactic agent to promote disease regression or inhibit the development or recurrence of the disease can be evaluated using a variety of methods known to the skilled practitioner, such as in human subjects during clinical trials, in animal model systems predictive of efficacy in humans, or by assaying the activity of the agent in in vitro assays.

Pharmaceutical compositions can be administered with medical devices known in the art. For example, a therapeutic composition of the invention can be administered with a needleless hypodermic injection device, such as the devices disclosed in U.S. Pat. Nos. 5,399,163, 5,383,851, 5,312,335, 5,064,413, 4,941,880, 4,790,824, and 4,596,556. Examples of well-known implants and modules useful in the present invention include those described in U.S. Pat. Nos. 4,487,603, 4,486,194, 4,447,233, 4,447,224, 4,439,196, and 4,475,196. These patents are incorporated herein by reference. Many other such implants, delivery systems, and modules are known to those skilled in the art.

In some embodiments, the method of treating a disease or disorder in a subject, as described above, further comprises administering to the subject an additional therapeutic agent or therapy. In some embodiments, The TPP1 protein may be used alone or with other active ingredients. It may be conjugated to a polyalkylene glycol moiety by known methods or may be used as part of a chimeric protein, for example, as linked to an antibody or parts thereof, a transferrin, a hormone, or a growth factor. The TPP1 protein may be provided in the form of a prodrug, i.e., a stable inactive form that becomes active once it is administered (for example, as described above). The instant invention provides for conjugating targeting molecules to TPP1, DNA vectors (including viruses) encoding TPP1, and carriers (i.e., liposomes) for targeting a desired cell or tissue, e.g., the brain. “Targeting molecule” as used herein shall mean a molecule which, when administered in vivo, localizes to desired location(s). In various embodiments, the targeting molecule can be a peptide or protein, antibody, lectin, carbohydrate, or steroid. In one embodiment, the targeting molecule is a protein or peptide ligand of an internalized receptor on the target cell. In a specific embodiment, the targeting molecule is a peptide comprising the well-known RGD sequence, or variants thereof that bind RGD receptors on the surface of cells such as cancer cells, e.g., human ova that have receptors that recognize the RGD sequence. Other ligands include, but are not limited to, transferrin, insulin, amylin, and the like. Receptor internalization is preferred to facilitate intracellular delivery of TPP1 protein. In another embodiment, the targeting molecule is an antibody. Preferably, the targeting molecule is a monoclonal antibody. In one embodiment, to facilitate crosslinking, the antibody can be reduced to two heavy and light chain heterodimers or the F(ab)₂fragment can be reduced and crosslinked to the TPP1 via the reduced sulfhydryl. Antibodies for use as targeting molecules are specific for cell surface antigen. In one embodiment, the antigen is a receptor. For example, an antibody specific for a receptor on cancer cells, such as melanoma cells, can be used. This invention further provides for the use of other targeting molecules, such as lectins, carbohydrates, proteins, and steroids.

A composition of this invention preferably includes an uptake inhibitor which decreases local clearance of TPP1 protein by cell surface receptors. This helps ensure that the TPP1 protein is administered evenly, such that more cells get some TPP1 protein, rather than the few cells close to the site of administration getting most of the TPP1 protein. Clearance mechanisms include endocytosis by cell surface receptors such as the mannose receptor, the asialoglycoprotein receptor, and the mannose-6-phosphate receptor. Thus a preferred uptake inhibitor is mannose-6-phosphate. The uptake inhibitor can be administered in a composition with TPP1 protein, or can be separately but simultaneously administered, or the two can be administered at different times as long as the uptake inhibitor is able to have the desired effect.

As used herein, “treatment” or “treating,” or “palliating” or “ameliorating” are used interchangeably. These terms refer to an approach for obtaining beneficial or desired results, including but not limited to a therapeutic benefit and/or a prophylactic benefit. By therapeutic benefit is meant any therapeutically relevant improvement in or effect on one or more diseases (e.g., inflammatory diseases), conditions, or symptoms under treatment. For prophylactic benefit, the agent or the compositions thereof may be administered to a subject at risk of developing a particular disease, condition, or symptom, or to a subject reporting one or more of the physiological symptoms of a disease, even though the disease, condition, or symptom may not have yet been manifested.

As used herein, the term “co-administration” or “co-administered” refers to the administration of at least two agent(s) or therapies to a subject. In some embodiments, the co-administration of two or more agents/therapies is concurrent. In other embodiments, a first agent/therapy is administered prior to a second agent/therapy. Those of skill in the art understand that the formulations and/or routes of administration of the various agents/therapies used may vary.

In many embodiments, the terms “subject” and “patient” are used interchangeably irrespective of whether the subject has or is currently undergoing any form of treatment. As used herein, the terms “subject” and “subjects” may refer to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgus monkey, chimpanzee, etc.) and a human). The subject may be a human or a non-human. In more exemplary aspects, the mammal is a human. As used herein, the expression “a subject in need thereof” or “a patient in need thereof” means a human or non-human mammal that exhibits one or more symptoms or indications of a disease or disorder and/or who has been diagnosed with a disease or disorder. In some embodiments, the subject is a mammal. In some embodiments, the subject is human.

“Sample,” “test sample,” and “patient sample” may be used interchangeably herein. The sample can be a sample of serum, urine plasma, amniotic fluid, cerebrospinal fluid, cells, or tissue. Such a sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art. The terms “sample” and “biological sample” as used herein generally refer to a biological material being tested for and/or suspected of containing an analyte of interest, such as antibodies. The sample may be any tissue sample from the subject. The sample may comprise protein from the subject.

The terms “increased,” “increase,” “elevate,” “enhance,” or “activate” are all used herein to generally mean an increase by a statically significant amount; for the avoidance of any doubt, the terms “increased,” “increase” or “enhance” or “activate” means an increase of at least 10% as compared to a reference level, for example, an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

### Animal Model for Studying Lysosomal Storage Diseases

In one aspect, this disclosure provides an animal model for studying a disease or disorder. In some embodiments, the animal model comprises: (i) a Tpp1 gene heterozygous knockout (Tpp1^{+/-}), and (ii) a Cln3 gene homozygous knockout (Cln3^{-/-}), wherein the mouse model has a shortened lifespan compared to a wild type animal.

A “gene” refers to a DNA sequence that encodes proteins and may or may not include introns, exons, regulatory sequences such as promoter or enhancer sequences and 5′ untranslated regions. A “transcript,” as referred to herein, is an RNA molecule derived by the transcription of a coding gene or nucleic acid.

The term “knock-out” in relation to a gene refers to the alteration of the wild type sequence of the gene such that no functional gene product is produced. The term “gene product” encompasses transcripts of the gene as well as proteins translated from said transcripts. For example, mutation, insertion or deletion of nucleotides of the wild type gene sequence may lead to the entire silencing of the gene's expression, or to the expression of the altered gene sequence such that only a non-functional transcript is produced. A non-functional transcript cannot be translated into a functional protein. If only one allele of the gene has been altered, the gene knocked out is referred to as a “heterozygous knock-out.” If both alleles have been altered, the knock-out is referred to as a “homozygous knock-out.” In accordance with convention in the field, a heterozygous gene knock-out is indicated by “het” or “+/−,” whereas a homozygous knock-out is indicated by “horn” or “−/−.” If both alleles of the gene remain unaltered (i.e., having the wild type gene sequence) it is indicated by “wt” or “++.”

The term “heterozygous” means a genetic condition existing when different alleles reside at corresponding loci on homologous chromosomes. The term “homozygous” means a genetic condition existing when identical alleles reside at corresponding loci on homologous chromosomes.

In some embodiments, the animal may, for example, be a mammal, including but not limited to a mouse, a rat, a sheep, a dog, a cow, a horse, a non-human primate, a pig, a cat, a rabbit, a goat, a ferret, a guinea pig, a gerbil or a hamster. The animal may also be a bird, including, but not limited to, a chicken, a duck or a quail. In some embodiments, the animal is a member of the Murine family. In a further embodiment, the animal is a mouse.

In some embodiments, the disease or disorder is selected from Late-infantile neuronal ceroid lipofuscinosis (CLN2) caused by mutations in the Tpp1 gene, Juvenile neuronal ceroid lipofuscinosis (CLN3) caused by mutations in the Cln3 gene, Variant late infantile neuronal ceroid lipofuscinosis type 5 (CLN5) disease caused by mutations in the Cln5 gene, Variant late infantile neuronal ceroid lipofuscinosis type 6 (CLN6) disease caused by mutations in the Cln6 gene, Neuronal ceroid lipofuscinosis type 7 (CLN7) disease caused by mutations in the MFSD8 gene, Northern epilepsy neuronal ceroid lipofuscinosis type 8 (CLN8) disease caused by mutations in the Cln8 gene, Congenital neuronal ceroid lipofuscinosis type 10 (CLN10) disease caused by mutations in the CTSD gene, Late-onset Neuronal ceroid lipofuscinosis (CLN12) and Kufor-Rakeb syndrome caused my mutations in the gene ATP13A2, Sanfilippo D syndrome (mucopolysaccharidosis type HID) caused by mutations in the GNS gene, and Osteopetrosis autosomal recessive 4 (OPTB4)caused by mutations in the CLCN7 gene.

In some embodiments, the animal has at least 25% (e.g., 25%, 30%, 35%, 40%, 45%, 50%, 55%, 60%, 65%, 70%, 75%, 80%) reduction in lifespan compared to the wild type animal.

In some embodiments, the Tpp1 gene or the Cln3 gene comprises at least one mutation selected from a deletion, an insertion, a frame-shift mutation, re-arrangement or a substitution. In some embodiments, the mutation is constitutive. In some embodiments, the mutation is conditional.

In some embodiments, the Tpp1 gene is located at Chr 7 E3; 7 55.97 cM. In some embodiments, the Tpp1 gene comprises a deletion of at least a portion of an exon within the Tpp1 gene. In some embodiments, the Tpp1 gene comprises an insertion of neo into intron 11 and an Arg446His missense mutation into exon 11 immediately upstream of the neo insertion.

In some embodiments, wherein the Cln3 gene is located at Chr 7 F3; 7 69.16 cM. In some embodiments, the Cln3 gene comprises a deletion of at least a portion of an exon within the Cln3 gene. In some embodiments, the Cln3 gene comprises a deletion of all or part of exons 1-6 within the Cln3 gene.

In some embodiments, the animal model has an increased level of lysosomal accumulation of subunit c of mitochondrial ATP synthase (SCMAS). In some embodiments, the animal model has at least 10%, 20%, 30%, 40%, 50%, 60%, 70%, 80%, 90%, 100%, 120%, 140%, 160%, 180%, 200% or more increase in the level of lysosomal accumulation of SCMAS. In some embodiments, the animal model has at least 50% increase in the level of lysosomal accumulation of SCMAS.

In some embodiments, the animal model has an increased expression level of NPC1 and/or CTSF. In some embodiments, the animal model has at least 40% or more increase in the expression level of NPC1 and/or CTSF, or at least 40% or more decrease in the expression level of SMPD1.

In some embodiments, the animal model has at least 40% increase in the expression level of NPC1 and/or CTSF, or 40% decrease in the expression level of SMPD1.

In some embodiments, the animal model is characterized by a deficit in a locomotor activity.

Also within the scope of this disclosure is a progeny of the animal model disclosed herein, and a cell, tissue, or cell line derived from the animal model or the progeny, as disclosed herein.

The term “progeny” refers to the descendant(s) of a particular cross. Typically, progeny result from breeding of two individuals. The descendant(s) can be, for example, of the F1, the F2, or any subsequent generation.

In another aspect, this disclosure also provides a method of obtaining the animal model disclosed above. The method comprises: (a) cross-breeding an animal with a Tpp1 knockout with a second animal with a Cln3 knockout to obtain an animal with double heterozygotes (Tpp1^{+/-}; Cln3^{+/-}); and (b) cross-breeding the animal with double heterozygotes by mating Tpp1^{-/-}; Cln3^{-/-}×Tpp1^{-/-}; Cln3^{-/-} or Tpp1^{-/-}; Cln3^{-/+}×Tpp1^{+/-}; Cln3^{-/-}.

### Methods of Identifying Agents for Treating Lysosomal Storage Diseases

In another aspect, this disclosure further provides a method of identifying an agent for use in treatment of a disease or disorder in a subject. In some embodiments, the method comprises administering a candidate agent to the animal model or the progeny, as disclosed herein, and assessing an effect of the candidate agent on a phenotype of the animal model.

In some embodiments, the method comprises contacting the cell, tissue, or cell line, as disclosed herein, with a candidate agent, and assessing an effect of the candidate agent on the cell, tissue, or cell line.

In some embodiments, the phenotype is the lifespan of the animal model.

In some embodiments, the effect is characterized by an increase in the lifespan of the animal model. In some embodiments, the effect is characterized by a decrease in the level of lysosomal accumulation of SCMAS. In some embodiments, the effect is characterized by a decrease in the expression level of NPC1 and/or CTSF.

In some embodiments, the candidate agent comprises a protein, a peptide, a peptidomimetic, a nucleic acid, or a small molecule.

In yet another aspect, this disclosure additionally provides a method for identifying an agent with TPP1 agonist activity. In some embodiments, the method comprises: (a) administering an amount of a candidate agent to a subject, e.g., the animal model disclosed herein; (b) performing an assay on a sample obtained from the subject and determining the level of lysosomal accumulation of SCMAS and/or the activity/level of TPP1 in the sample; and (c) identifying the agent having TPP1 agonist activity if the subject has a reduced level of lysosomal accumulation of SCMAS and/or increased activity/level of TPP1 as compared to a reference level.

In some embodiments, the method comprises: (a) contacting a candidate agent with a sample comprising the cell, tissue, or cell line, as disclosed herein; (b) performing an assay on the sample and determining the level of lysosomal accumulation of SCMAS and/or the activity/level of TPP1 in the sample; and (c) identifying the agent having TPP1 agonist activity if the sample exhibits a reduced level of lysosomal accumulation of SCMAS and/or increased activity/level of TPP1 as compared to a reference level.

In some embodiments, the method may include culturing or expanding a test cell and/or a control cell. The term “culturing” or “expanding” refers to maintaining or cultivating cells under conditions in which they can proliferate and avoid senescence. For example, cells may be cultured in media optionally containing one or more growth factors, i.e., a growth factor cocktail. In some embodiments, the cell culture medium is a defined cell culture medium. The cell culture medium may include neoantigen peptides. Stable cell lines may be established to allow for the continued propagation of cells.

In some embodiments, the reference level may be obtained from the subject prior to the administration of the agent or from a cell prior to contacting with the agent, for increasing a level or activity of TPP1 or a composition thereof. In some embodiments, the reference level may be obtained from a control subject or a group of individuals who do not have a disease or disorder or have not been diagnosed with a disease or disorder, or from a control cell or an unaffected cell in the subject. In some embodiments, the reference level is obtained based on average levels of the level or activity of TPP1, for example, of a population not suffering from a disease or disorder. In some embodiments, the reference level is obtained based on a median or median level of a set of individuals in which patients with a disease or disorder are included.

In some embodiments, the disease or disorder is characterized by accumulation of one or more storage products (e.g., SCMAS) in the lysosomes of the affected cells, such as neurons. One mode of determining the disorder is finding that the lysosomes have accumulated storage material, which can be done by known methods such as microscopy or immunofluorescence. An example of a storage material that would be detected in lysosomes is SCMAS. In some embodiments, treatment with TPP1 protein will reduce or eliminate mitochondrial ATP synthase, in particular SCMAS in the lysosomes of the affected cells, such as neurons. Detecting elimination of storage material such as mitochondria SCMAS in the lysosomes of affected cells can be done by known methods as described above.

In some embodiments, the disease or disorder is selected from Late-infantile neuronal ceroid lipofuscinosis (CLN2) caused by mutations in the Tpp1 gene, Juvenile neuronal ceroid lipofuscinosis (CLN3) caused by mutations in the Cln3 gene, Variant late infantile neuronal ceroid lipofuscinosis type 5 (CLN5) disease caused by mutations in the Cln5 gene, Variant late infantile neuronal ceroid lipofuscinosis type 6 (CLN6) disease caused by mutations in the Cln6 gene, Neuronal ceroid lipofuscinosis type 7 (CLN7) disease caused by mutations in the MFSD8 gene, Northern epilepsy neuronal ceroid lipofuscinosis type 8 (CLN8) disease caused by mutations in the Cln8 gene, Congenital neuronal ceroid lipofuscinosis type 10 (CLN10) disease caused by mutations in the CTSD gene, Late-onset Neuronal ceroid lipofuscinosis (CLN12) and Kufor-Rakeb syndrome caused my mutations in the gene ATP13A2, Sanfilippo D syndrome (mucopolysaccharidosis type IID) caused by mutations in the GAS gene, and Osteopetrosis autosomal recessive 4 (OPTB4)caused by mutations in the CLCN7 gene.

In some embodiments, the disease or disorder is characterized by a deficiency in a function of a CLN3 protein. An example of such a disorder is JNCL.

In some embodiments, the affected cells may belong to any cell or tissue type, such as neurons. In some embodiments, the affected cells are neuronal cells.

The level or activity of TPP1 may be measured by determining or estimating a protein level or mRNA level. Methods for determining or estimating a protein level or mRNA level are well known in the art. Such methods may include enzyme activity assays, microscopy, immunofluorescence, and nucleic acid hybridization (e.g., using proteins and nucleic acids described in U.S. Ser. No. 08/931,608 and Sleat et al. (1997)). For example, the protein level (e.g., protein expression level) of TPP1 can be determined by SDS-PAGE, Western blot, or an immunoassay (e.g., immunoblotting assay, immunoprecipitation assay). The mRNA level may be determined by RT-PCR.

Measuring the levels of TPP1 can be performed by assaying the proteins themselves (by Western blotting, ELISA, RIA, and other techniques known to one skilled in the art), by assaying the mRNA encoding these proteins (such as quantitative PCR, Northern blotting, RNAse protection assay, RNA dot-blotting, and other techniques known to one skilled in the art), or by assaying the activity of the regulatory elements of the genes for TPP1. For example, the activity of regulatory elements can be assessed by reporter constructs consisting of DNA segments from the promoter, enhancer, and/or intronic elements coupled to cDNAs encoding reporters (such as luciferase, beta-galactosidase, green fluorescent protein, or other reporting genes that can be easily assayed). These reporter constructs can be transfected into cells, either stably or transiently.

### Additional Definitions

To aid in understanding the detailed description of the compositions and methods according to the disclosure, a few express definitions are provided to facilitate an unambiguous disclosure of the various aspects of the disclosure. Unless otherwise defined, all technical and scientific terms used herein have the same meaning as commonly understood by one of ordinary skill in the art to which this disclosure belongs.

The term “agent” is used herein to denote a chemical compound, a mixture of chemical compounds, a biological macromolecule (such as a nucleic acid, an antibody, a protein or portion thereof, e.g., a peptide), or an extract made from biological materials such as bacteria, plants, fungi, or animal (particularly mammalian) cells or tissues. The activity of such agents may render it suitable as a “therapeutic agent,” which is a biologically, physiologically, or pharmacologically active substance (or substances) that acts locally or systemically in a subject.

The terms “therapeutic agent,” “therapeutic capable agent,” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder, or condition; and generally counteracting a disease, symptom, disorder or pathological condition.

The terms “polypeptide,” “peptide,” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, pegylation, or any other manipulation, such as conjugation with a labeling component. As used herein, the term “amino acid” includes natural and/or unnatural or synthetic amino acids, including glycine and both the D or L optical isomers, and amino acid analogs and peptidomimetics.

A “nucleic acid” or “polynucleotide” refers to a DNA molecule (for example, but not limited to, a cDNA or genomic DNA) or an RNA molecule (for example, but not limited to, an mRNA) and includes DNA or RNA analogs. A DNA or RNA analog can be synthesized from nucleotide analogs. The DNA or RNA molecules may include portions that are not naturally occurring, such as modified bases, modified backbone, deoxyribonucleotides in an RNA, etc. The nucleic acid molecule can be single-stranded or double-stranded.

The term “operably linked” refers to a functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary, to join two protein-coding regions, in the same reading frame.

The term “linker” refers to any means, entity, or moiety used to join two or more entities. A linker can be a covalent linker or a non-covalent linker. Examples of covalent linkers include covalent bonds or a linker moiety covalently attached to one or more of the proteins or domains to be linked. The linker can also be a non-covalent bond, e.g., an organometallic bond through a metal center such as a platinum atom. For covalent linkages, various functionalities can be used, such as amide groups, including carbonic acid derivatives, ethers, esters, including organic and inorganic esters, amino, urethane, urea, and the like. To provide for linking, the domains can be modified by oxidation, hydroxylation, substitution, reduction etc., to provide a site for coupling. Methods for conjugation are well known by persons skilled in the art and are encompassed for use in the present invention. Linker moieties include, but are not limited to, chemical linker moieties, or for example, a peptide linker moiety (a linker sequence).

By “isolated” nucleic acid molecule or polynucleotide is intended as a nucleic acid molecule, DNA or RNA, which has been removed from its native environment. For example, a recombinant polynucleotide encoding a therapeutic polypeptide contained in a vector is considered isolated for the purposes of the present invention. Further examples of an isolated polynucleotide include recombinant polynucleotides maintained in heterologous host cells or purified (partially or substantially) polynucleotides in solution. An isolated polynucleotide includes a polynucleotide molecule contained in cells that ordinarily contain the polynucleotide molecule, but the polynucleotide molecule is present extrachromosomally or at a chromosomal location that is different from its natural chromosomal location. Isolated RNA molecules include in vivo or in vitro RNA transcripts of the present invention, as well as positive and negative strand forms, and double-stranded forms. Isolated polynucleotides or nucleic acids may further include such molecules produced synthetically. In addition, a polynucleotide or a nucleic acid may be or may include a regulatory element such as a promoter, ribosome binding site, or a transcription terminator.

The term “substantial identity” or “substantially identical,” when referring to a nucleic acid or fragment thereof, indicates that, when optimally aligned with appropriate nucleotide insertions or deletions with another nucleic acid (or its complementary strand), there is nucleotide sequence identity in at least about 90%, and more preferably at least about 95%, 96%, 97%, 98% or 99% of the nucleotide bases, as measured by any well-known algorithm of sequence identity, such as FASTA, BLAST or GAP, as discussed below. A nucleic acid molecule having substantial identity to a reference nucleic acid molecule may, in certain instances, encode a polypeptide having the same or substantially similar amino acid sequence as the polypeptide encoded by the reference nucleic acid molecule.

As applied to polypeptides, the term “substantial similarity” or “substantially similar” means that two peptide sequences, when optimally aligned, such as by the programs GAP or BESTFIT using default gap weights, share at least 90% sequence identity, even more preferably at least 95%, 98% or 99% sequence identity. Preferably, residue positions, which are not identical, differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent or degree of similarity may be adjusted upwards to correct for the conservative nature of the substitution. Means for making this adjustment are well known to those of skill in the art. See, e.g., Pearson (1994) Methods Mol. Biol. 24: 307-331, which is herein incorporated by reference.

The term “disease” as used herein is intended to be generally synonymous and is used interchangeably with, the terms “disorder” and “condition” (as in medical condition), in that all reflect an abnormal condition (e.g., disease or disorder) of the human or animal body or of one of its parts that impairs normal functioning, is typically manifested by distinguishing signs and symptoms, and causes the human or animal to have a reduced duration or quality of life.

As used herein, the term “modulate” is meant to refer to any change in biological state, i.e., increasing, decreasing, and the like.

As used herein, the term “pharmaceutically acceptable” refers to a material, such as a carrier or diluent, which does not abrogate the biological activity or properties of the composition, and is relatively non-toxic, i.e., the material may be administered to an individual without causing undesirable biological effects or interacting in a deleterious manner with any of the components of the composition in which it is contained.

The term “pharmaceutically acceptable carrier” includes a pharmaceutically acceptable salt, pharmaceutically acceptable material, composition or carrier, such as a liquid or solid filler, diluent, excipient, solvent or encapsulating material, involved in carrying or transporting a compound(s) of the present invention within or to the subject such that it may perform its intended function. Typically, such compounds are carried or transported from one organ, or portion of the body, to another organ, or portion of the body. Each salt or carrier must be “acceptable” in the sense of being compatible with the other ingredients of the formulation, and not injurious to the subject. Some examples of materials that may serve as pharmaceutically acceptable carriers include: sugars, such as lactose, glucose, and sucrose; starches, such as corn starch and potato starch; cellulose, and its derivatives, such as sodium carboxymethyl cellulose, ethyl cellulose, and cellulose acetate; powdered tragacanth; malt; gelatin; talc; excipients, such as cocoa butter and suppository waxes; oils, such as peanut oil, cottonseed oil, safflower oil, sesame oil, olive oil, corn oil, and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; diluent; granulating agent; lubricant; binder; disintegrating agent; wetting agent; emulsifier; coloring agent; release agent; coating agent; sweetening agent; flavoring agent; perfuming agent; preservative; antioxidant; plasticizer; gelling agent; thickener; hardener; setting agent; suspending agent; surfactant; humectant; carrier; stabilizer, and other non-toxic compatible substances employed in pharmaceutical formulations, or any combination thereof. As used herein, “pharmaceutically acceptable carrier” also includes any and all coatings, antibacterial and antifungal agents, absorption delaying agents, and the like that are compatible with the activity of one or more components of the invention, and are physiologically acceptable to the subject. Supplementary active compounds may also be incorporated into the compositions.

As used herein, the term “in vitro” refers to events that occur in an artificial environment, e.g., in a test tube or reaction vessel, in cell culture, etc., rather than within a multi-cellular organism.

As used herein, the term “in vivo” refers to events that occur within a multi-cellular organism, such as a non-human animal.

It is noted here that, as used in this specification and the appended claims, the singular forms “a,” “an,” and “the” include plural reference unless the context clearly dictates otherwise.

The terms “including,” “comprising,” “containing,” or “having” and variations thereof are meant to encompass the items listed thereafter and equivalents thereof as well as additional subject matter unless otherwise noted.

The phrases “in one embodiment,” “in various embodiments,” “in some embodiments,” and the like are used repeatedly. Such phrases do not necessarily refer to the same embodiment, but they may unless the context dictates otherwise.

The terms “and/or” or “/” means any one of the items, any combination of the items, or all of the items with which this term is associated.

The word “substantially” does not exclude “completely,” e.g., a composition which is “substantially free” from Y may be completely free from Y. Where necessary, the word “substantially” may be omitted from the definition of the invention.

As used herein, the term “approximately” or “about,” as applied to one or more values of interest, refers to a value that is similar to a stated reference value. In some embodiments, the term “approximately” or “about” refers to a range of values that fall within 25%, 20%, 19%, 18%, 17%, 16%, 15%, 14%, 13%, 12%, 11%, 10%, 90%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Unless indicated otherwise herein, the term “about” is intended to include values, e.g., weight percents, proximate to the recited range that are equivalent in terms of the functionality of the individual ingredient, the composition, or the embodiment.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

As used herein, the term “each,” when used in reference to a collection of items, is intended to identify an individual item in the collection but does not necessarily refer to every item in the collection. Exceptions can occur if explicit disclosure or context clearly dictates otherwise.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention. When used in this document, the term “exemplary” is intended to mean “by way of example” and is not intended to indicate that a particular exemplary item is preferred or required.

All methods described herein are performed in any suitable order unless otherwise indicated herein or otherwise clearly contradicted by context. In regard to any of the methods provided, the steps of the method may occur simultaneously or sequentially. When the steps of the method occur sequentially, the steps may occur in any order, unless noted otherwise.

In cases in which a method comprises a combination of steps, each and every combination or sub-combination of the steps is encompassed within the scope of the disclosure, unless otherwise noted herein.

Each publication, patent application, patent, and other reference cited herein is incorporated by reference in its entirety to the extent that it is not inconsistent with the present disclosure. Publications disclosed herein are provided solely for their disclosure prior to the filing date of the present invention. Nothing herein is to be construed as an admission that the present invention is not entitled to antedate such publication by virtue of prior invention. Further, the dates of publication provided may be different from the actual publication dates, which may need to be independently confirmed.

It is understood that the examples and embodiments described herein are for illustrative purposes only and that various modifications or changes in light thereof will be suggested to persons skilled in the art and are to be included within the spirit and purview of this application and scope of the appended claims.

### EXAMPLES

### Example 1

This example describes the materials and methods used in subsequent EXAMPLES below.

### Animals

Mice were maintained and used following protocols approved by the Rutgers University and Robert Wood Johnson Medical School Institutional Animal Care and Use Committee (“Preclinical evaluation of therapy in an animal model for LINCL,” protocol I09-0274-4). Tpp1^{-/-} and Cln3^{-/-} mice were in a C57BL/6 genetic background, and genotyping was conducted as described (Sleat D E, et al. J Neurosci 24:9117-26 (2004); Greene N D, et al. Mol Genet Metab 66:309-13 (1999)). Experimental cohorts contained equal numbers of male and female animals and were analyzed at ˜120 days of age. For biochemical analyses, animals were deeply anesthetized with sodium pentobarbital/phenytoin (a 1:4 dilution of Euthasol; Delmarva Laboratories, Midlothian, VA) and euthanized by transcardial perfusion with 0.9% saline. Brains were dissected and frozen on dry ice. For histopathology, mice were anesthetized, perfused with saline, then perfusion-fixed with 4% paraformaldehyde in PBS. Brains were excised, fixed for 48 hrs in 4% paraformaldehyde in PBS, and then transferred to 30% sucrose/PBS at 4° C. until they sunk.

### Quantitative Mass Spectrometry

Sample preparation and quantitative mass spectrometry on whole-brain extracts was conducted as described previously (Sleat D E, et al. Mol Cell Proteomics 18:2244-2261 (2019)). In brief, detergent-solubilized extracts were prepared, proteins were digested in-solution sequentially with trypsin (specificity, carboxyl side of K and R) and endoprotease LysC (specificity, carboxyl side of K), and the resulting peptides were labeled with TMT11-plex isobaric reagents (ThermoFisher Scientific). Labeled samples were pooled and analyzed by synchronous precursor selection MS3 on a Lumos Tribrid instrument (ThermoFisher Scientific). Peak lists were generated using Proteome Discoverer 2.2 (ThermoFisher Scientific), and data were searched using a local implementation of the Global Proteome Machine (GPM) (Craig R, Cortens J P and Beavis R C (2004) J Proteome Res 3:1234-42; Beavis R C (2006) Methods Mol Biol 328:217-28). Reporter ion intensities were extracted using in-house scripts (https://github.com/cgermain/IDEAA). Mass spectrometry files (mgf and raw files, GPM search files, and Excel files denoting protein assignments, peptide-spectrum matches, and corresponding reporter ion intensities) are archived in the MassIVE (http://massive.ucsd.edu) and ProteomeXchange (http://www.proteomexchange.org/) repositories in submission MSV000087613.

### Normalization and Statistical Analyses of Mass Spectrometry Data

TMT-16 reporter ion intensities were normalized and analyzed as described previously (Sleat D E, et al. (2017) J Proteome Res 16:3787-3804). In brief, reporter ion intensity data were extracted from peak list files using custom in-house scripts (https://github.com/cgermain/IDEAA), then spectra were normalized to total reporter ion intensity per channel to correct for differences in labeling efficiency and/or amounts of protein labeled. Peptides were first filtered for fully tryptic cleavage, no missed cleavage sites, and complete iTRAQ labeling of lysines and amino termini. Peptides were then filtered to remove those containing posttranslational modifications that may increase variability in the data (i.e., asparagine or glutamine deamidation, methionine dioxidation, tryptophan mono- and dioxidation, and isobaric labeling of tyrosine at positions other than the amino terminus). For selected comparisons, ratios of expression and q-values corrected for multiple comparisons using the Benjamini-Hochberg procedure were generated using a nested procedure that accounts for variability at both peptide and protein levels (Y B and Y H (1995) Journal of the Royal Statistical Society: Series B (Statistical Methodology) 57:289-300).

### Immunostaining

For immunohistochemistry, one in six series of coronal brain sections from each mouse were stained using a modified immunofluorescence protocol (Nelvagal H R, et al. Neuropathol Appl Neurobiol 47:251-267) for the astrocyte marker glial fibrillary associated protein (GFAP, rabbit anti-GFAP, 1:1000, DAKO), and the microglial marker CD68 (rat anti-mouse CD68, 1:400, Bio-Rad). Briefly, 40 μm coronal sections were mounted on Superfrost Plus slides (Fisher Scientific) and air-dried for 30 mins. Slides were then blocked in a 15% serum solution in 2% TBS-T (1× Tris Buffered Saline, pH 7.6 with 2% Triton-X100, Fisher Scientific) for 1 hour. Slides were then incubated in primary antibody in 10% serum solution in 2% TBS-T for 2 hours, washed three times in 1×TBS, and incubated with fluorescent Alexa-Fluor labeled IgG secondary antibodies (Alexa-Fluor goat anti-rabbit 488, goat anti-rat 546, Invitrogen) in 10% serum solution in 2% TBS-T for 2 hours. Slides were washed three times in 1×TBS and incubated in a 1× solution of TrueBlack lipofuscin autofluorescence quencher (Biotium, Fremont, CA) in 70% ethanol for 2 mins before rinsing with 1×TBS. Slides were cover-slipped in Fluoromount-G mounting medium with DAPI (Southern Biotech, Birmingham, AL).

### Thresholding Image Analysis

To analyze the degree of glial activation in the gray matter (GFAP positive astrocytes+CD68 positive microglia), a semiautomated thresholding image analysis method was used with Image-Pro Premier software (Media Cybernetics). Briefly, stained sections were scanned using a Zeiss AxioScan Z1 slide scanner at the Washington University Center for Cellular Imaging (WUCCI), at 10× magnification of each one in six series of sections per animal, followed by demarcation of all regions of interest. Images were subsequently analyzed using Image-Pro Premier (Media Cybernetics) using an appropriate threshold that selected the foreground immunoreactivity above the background. This threshold was then applied as a constant to all subsequent images analyzed per batch of animals and reagent used to determine the specific area of immunoreactivity for each antigen. Measurements for histological processing were performed blind to genotype, and statistical analyses were performed using GraphPad Prism version 8.0.0 for MacOS. Data were analyzed using two-way ANOVA with a post hoc Bonferroni correction, with a p-value of 50.05 considered significant.

### Survival Statistics

Survival curves were compared using log-rank tests in GraphPad Prism 9.1 (GraphPad Software, San Diego, California USA, www.graphpad.com) using the Bonferroni method to correct for multiple comparisons.

### Example 2

### Breeding Strategy

Initially, for generating all required genotypes as littermates, dihybrid crosses were conducted using animals that were double heterozygous for both Tpp1 and Cln3 mutations. Analysis of transmission ratios indicated that offspring were not generated in the predicted ratios (data not shown), with a marked reduction in the number of both double mutant and double wild type animals. This reflects gene linkage with both Tpp1 and Cln3 located on chromosome 7 in the mouse (Tpp1 Location: Chr 7 E3; 7 55.97 cM, Cln3 Location: Chr 7 F3; 7 69.16 cM). Subsequently, double knockout animals were generated by Tpp1^{-/-}; Cln3^{-/-}×Tpp1^{-/-}; Cln3^{-/-} or Tpp1^{-/-}; Cln3^{-/+}×Tpp1^{-/-}; Cln3^{-/-} matings.

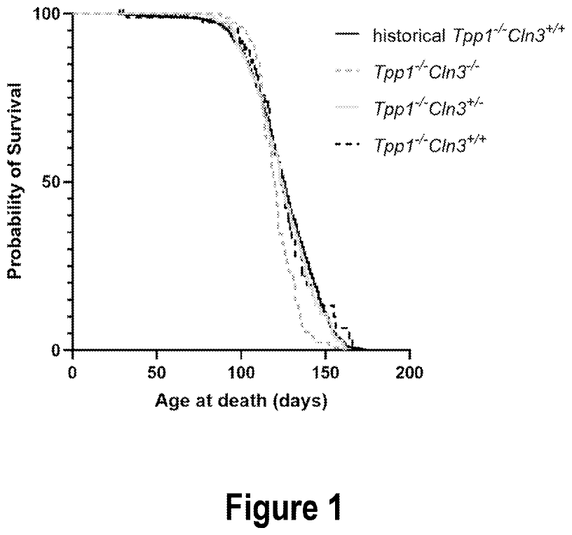

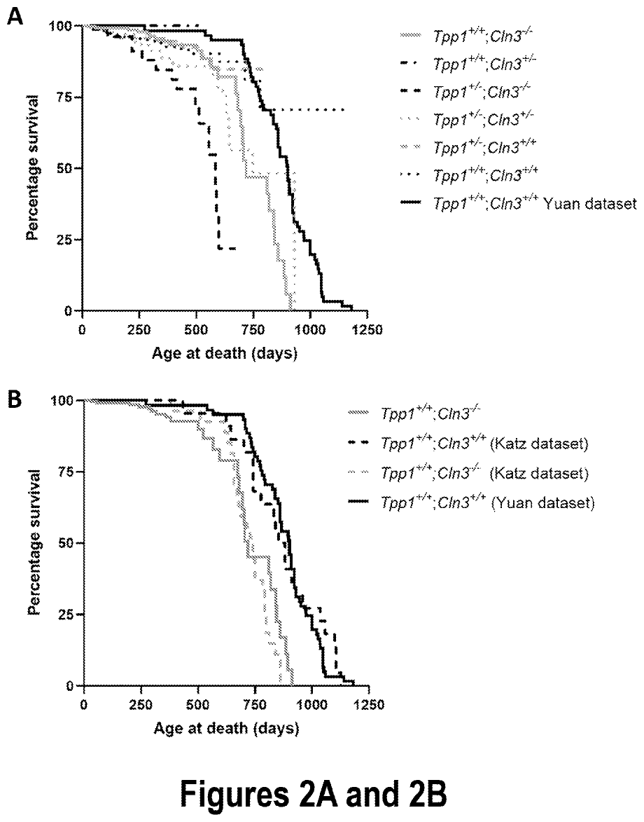

Survival Survival curves and statistical comparisons are shown in FIGS. 1 and 2, and Table 1. Given the substantial differences in survival, animals on a Tpp1^{-/-} background are shown in FIG. 1 and on a Tpp1^{+/-} or Tpp1^{+/+} background are shown in FIG. 2.

Tpp1^{-/-}; Cln3^{-/-}, Tpp1^{-/-}; Cln3⁺⁻, and Tpp1^{-/-}; Cln3^{+/+} littermates generated by double mutant matings had median survivals between 120-124 days (Table 1) and were not significantly different (after correction for multiple comparisons) from each other. However, the survival curve of Tpp1^{-/-}; Cln3^{-/-} mice is somewhat rectangularized compared to the Tpp1^{-/-}; Cln3^{+/+} mice. This may be an effect of the CLN3 mutation on the Tpp1^{-/-} a phenotype or alternatively, may reflect a subtle difference in the genetic background of the animals. The Tpp1 mutant allele used for these studies was backcrossed against C57BU6 (Sleat D E, et al. (2004) J Neurosci 24:9117-26) as was the Cln3 allele (Hersrud S L, et al. Biochim Biophys Acta 1862:1324-36). However, the strain background of the two mutants differs in that the Tpp1^{-/-} mice were Nnt^{+/+} while the Cln3^{-/-} mice were Nnt^{-/-} ((Sleat D E, et al. Mol Cell Proteomics 18:2244-2261). Mutations in Nnt vary in different C57BL/6 substrains. The Nnt^{-/-} genotype arose at Jackson Laboratories https://pubmed.ncbi.nlm.nih.gov/19448337/and is designated as C57BL/6J while Nnt^{+/+} strains are designated as C57BL/6N. As a result, double mutant animals were mixed C57BU6N×C57BL/6J substrains. There was no significant difference in survival between littermate Tpp1^{-/-}; Cln3^{+/+} mice generated from double Tpp1/Cln3 mutant matings and historical data from littermate Tpp1^{-/-}; Cln3^{+/+} generated by single Tpp1 mutant matings. Regardless, the primary conclusion is that there was no significant difference in survival phenotype of the Tpp1^{-/-}Cln3^{-/-} double mutant and Tpp1^{-/-}Cln3^{+/+} single mutant animals.

Survival of mutant animals in Tpp1^{-/-} or Tpp1^{+/+} backgrounds is shown in FIG. 2. Data from C57BL/6J animals from another study (“Yuan dataset”) (Yuan R, et al. (2012) Proc Natl Acad Sci USA 109:8224-9) is also included for analysis. Survival of Tpp1 heterozygous animals (median survival 843 days) did not differ significantly from the Yuan dataset wild type animals (median survival 901 days) (FIG. 2A). Given that heterozygosity for Tpp1 did not affect survival, no significant differences in survival of Cln3 heterozygotes were predicted when compared to wild type. Survival of Tpp1^{+/-}; Cln3^{+/-} double heterozygote mice (media 748 days) was similar to the single Cln3^{-/-} mice and was also significantly shorter than wild type animals. Tpp1 heterozygosity resulted in decreased survival of CLN3^{-/-} animals (median 584 days), which was significantly shorter than that of the Tpp1^{+/+}; Cln3^{-/-} animals. Overall, these data indicate that heterozygosity for Tpp1 resulted in decreased survival of both Cln3^{+/-} and Cln3^{-/-} animals.

Survival of our Tpp1^{+/+}; Cln3^{-/-} mice (median 719 days) was not significantly different from historical data from a different but similar JNCL knockout mouse model (742 days) (FIG. 2B). However, survival of both Cln3 mutants was significantly reduced compared to wild type mice.

### Pathology

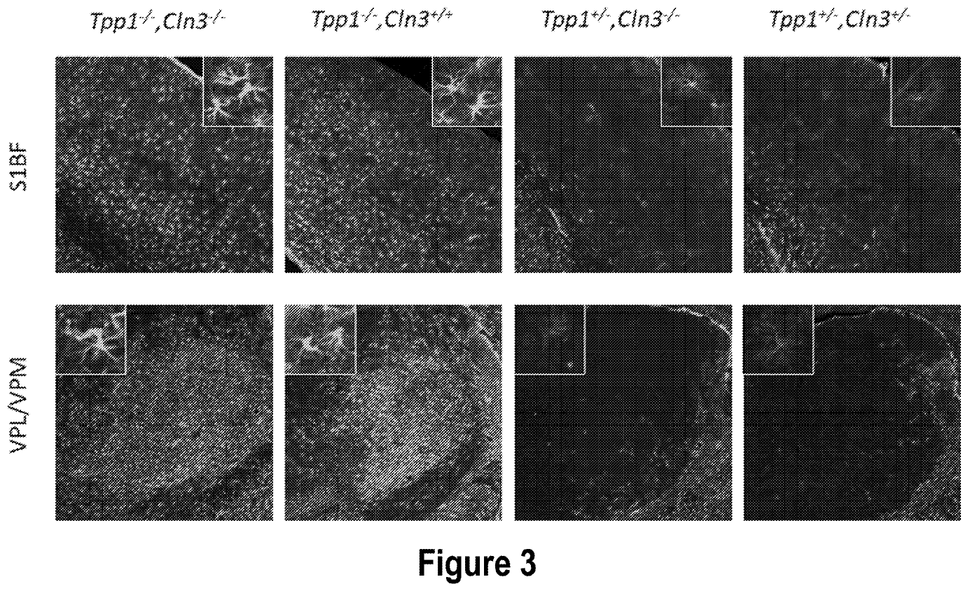

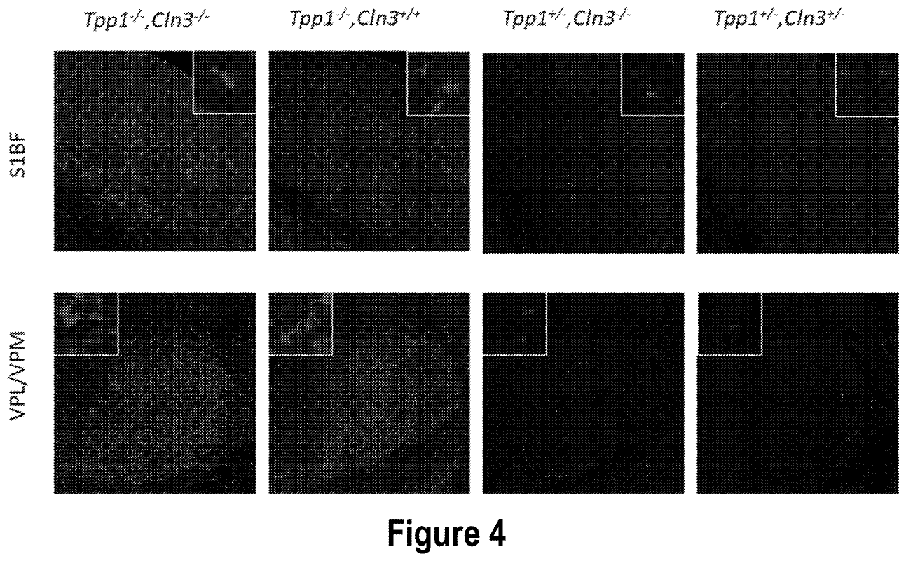

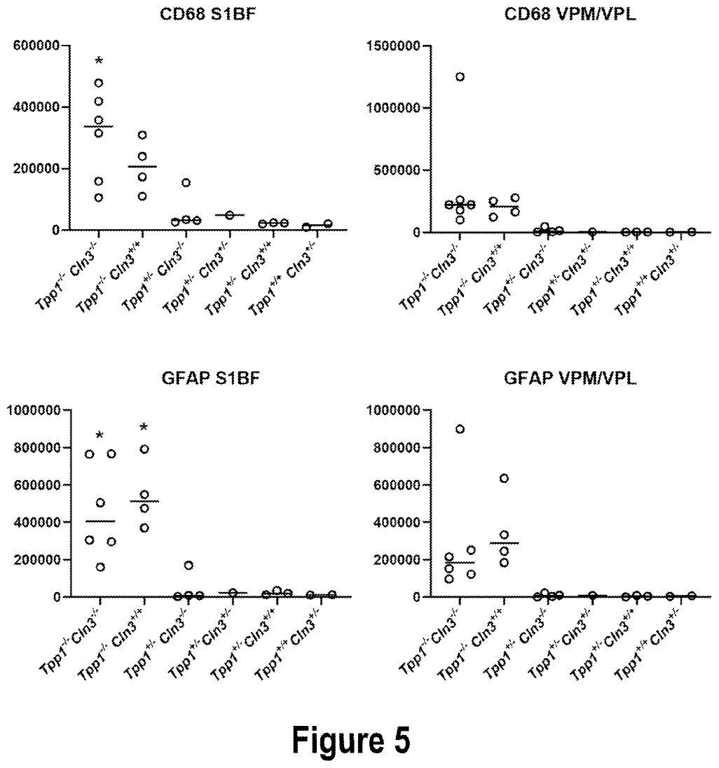

Animals were euthanized at ˜120 days to investigate the effect of Tpp1 and Cln3 genotype on brain pathology. Sections were analyzed by immunofluorescence for the simultaneous detection of markers of glial activation (CD68 in microglia and GFAP in astrocytes) in two brain regions where pronounced glial activation is consistently observed in multiple NCL mouse models: the somatosensory barrel field (S1BF) cortex and the medial and lateral ventral posterior nuclei of the thalamus (VPM/VPL) regions of the thalamus. As expected, immunostaining for either GFAP or CD68 was present at very low levels in animals with a Tpp1^{+/-} or Tpp1^{+/+} genotype, irrespective of Cln3 genotype. In these animals, only very faintly stained GFAP-positive astrocytes (FIG. 3) or CD68 positive microglia (FIG. 4) were present. Marked activation of both astrocytes (FIG. 3) and microglia (FIG. 4) was detected in the S1BF and VPM/VPL of Tpp1^{-/-}Cln3^{+/+} animals, with pronounced upregulation of these markers and corresponding changes in cellular morphology. Similar levels of pronounced glial activation were detected in the S1BF and VPM/VPL of double mutant Tpp1^{-/-}Cln3^{+/+} animals, with similar degrees of cellular hypertrophy of both astrocytes and microglia apparent to those seen in Tpp1^{-/-}Cln3^{+/+} animals. Thresholding image analysis confirmed these qualitative observations with significant elevation of both GFAP and CD68 immunoreactivity in both brain regions of Tpp1^{-/-}Cln3^{+/+} and Tpp1^{-/-}Cln3^{-/-} animals compared to other genotypes (FIG. 5). Although the individual levels of GFAP and CD68 immunoreactivity differed to some extent between Tpp1^{-/-}Cln3^{+/+} and Tpp1^{-/-}Cln3^{-/-} animals, there were no significant differences for either antigen in either brain region between animals of these genotypes. This broad similarity between Tpp1^{-/-}Cln3^{+/+} and Tpp1^{-/-}Cln3^{-/-} animals in terms of the extent of glial activation is consistent with the similarities observed in the survival phenotypes of these mutants.

### Proteomic Analysis

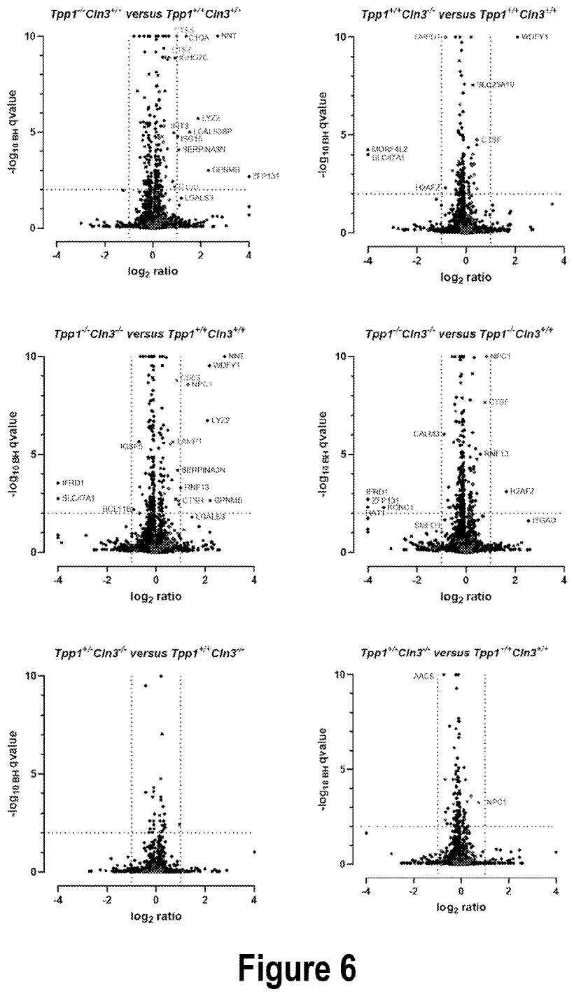

A proteomic analysis was conducted on brain samples from the Tpp1 and Cln3 mutant mice with the goal of identifying changes that might correlate with disease survival and may shed light on the respective cellular functions of these proteins. In addition, given that earlier proteomic studies failed to identify any informative brain expression changes in Cln3^{-/-} mice, it was hypothesized that changes related to Cln3 mutation could be exacerbated and thus possibly easier to detect in a Tpp1 mutant background. Proteins in brain extracts from animals at ˜105 days were identified and quantified using isobaric labeling mass spectrometry. In FIG. 6, various genotypes of interest were compared using volcano plots—a scatter plot that allows comparison of effect size with probability of significant difference. Q values (i.e., p-values adjusted for multiple comparisons found using the Benjamini-Hochberg false discovery rate method) were calculated by comparing reporter ion intensities for all individual spectra assigned to a given protein from all biological replicates for each genotype.

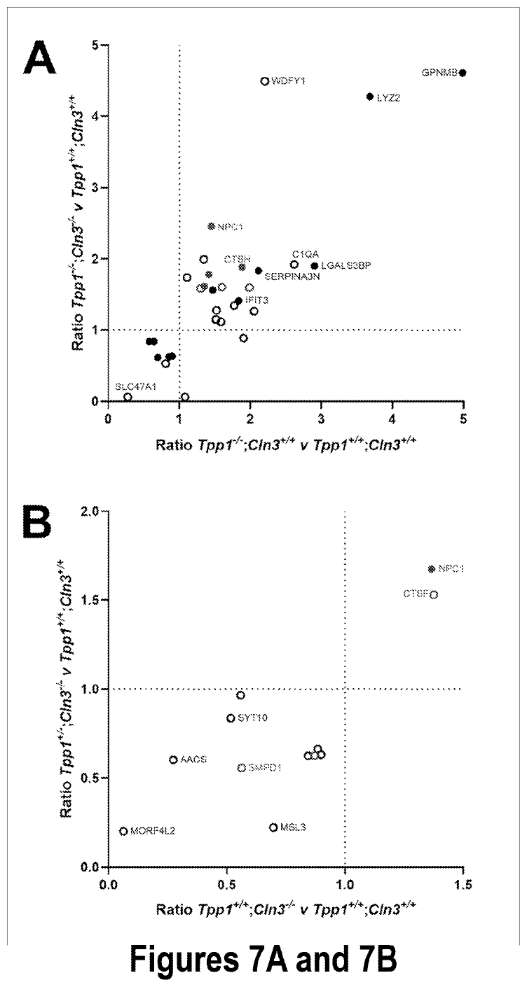

A number of significant changes in protein expression were detected when Tpp1^{-/-} animals were compared with wild type, including elevation of GPNMB, LYZ2, SERPINA3N, and various lysosomal proteins including several cathepsins (Table 2). When double mutant Tpp1^{-/-}; Cln3^{-/-} animals were compared to wild type, very similar changes in protein expression were identified for the most part, as illustrated by the high degree of correlation between fold changes in expression compared to wild type in Tpp1^{-/-}; Cln3^{+/+} and Tpp1^{-/-}; Cln3^{-/-} animals (FIG. 7A). Relatively few changes were detected in the Cln3^{-/-} mutant animals in Tpp1^{-/-} or Tpp1^{+/+} backgrounds, but NPC1 and CTSF were consistently elevated while NEU4 and SMPD1 were decreased (FIG. 7B).

### TPP1 Activity in Mutant Mice.

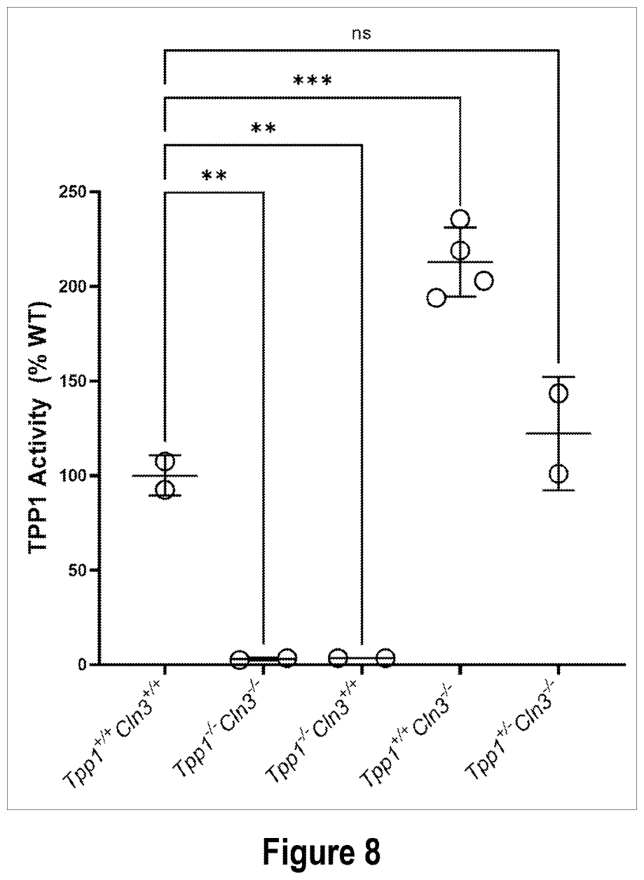

As shown in FIG. 8, it was found that TPP1 activity in the Cln3 mutant is ˜2-fold higher than that measured in wild type animals, while no activity was detectable in Tpp1 4 animals irrespective of Cln3 genotype. TPP1 activity in Tpp^{+/-} animals is, as expected, ˜50% of wild type, TPP1 activity if Tpp^{+/-} Cln3^{-/-} animals is ˜100% of wild type. This again indicates that loss of CLN3 results in an ˜2-fold increase in TPP1 activity.

The overall goal of this study was to determine the phenotype of a double Tpp1 and Cln3 mutant mouse model, which may provide useful information regarding the cellular role of CLN3 and potential functional interactions between TPP1 and CLN3. For the most part, the phenotype of these double knockout mice was similar or identical to the single Tpp1 mutant: median survival of both models is within five days, and they display similar extents of characteristic NCL pathology in the form of astrocytosis and microglial activation. In addition, proteomic changes within the brain of the double knockout are very similar to those in the single Tpp1 knockout (FIG. 7A). Consistent with these observations, one possibility is that the effects of disease in JNCL could result, at least in part, from secondary effects of CLN3 on TPP1. While loss of CLN3 does not appear to directly affect TPP1 activity measured in vitro, it is possible that the loss of CLN3 could affect access of TPP1 to its physiological substrates, possibly including SCMAS. Two observations provide support for this possibility. First, storage of SCMAS is present in both LINCL and JNCL (although it is also present in other lysosomal storage diseases). Second, an ˜2-fold increase was observed in TPP1 activity in the absence of CLN3 in this study (FIG. 8), indicating a compensatory response.

Lifespan of the JNCL mouse model (median 719 days) was shortened compared to wild type (median 901 days), and this is consistent with previous analysis of a different Cln3 knockout mouse model (Cln3^{-/-}, median 721 days, wild type median 861 days). However, heterozygosity for Tpp1 was associated with shortened survival of Cln3^{-/-} mice, further decreasing lifespan to a median of 584 days. In addition, survival of a double heterozygote Tpp1^{+/-}; Cln3^{+/-} was also significantly reduced compared to wild type. It is not clear why heterozygosity for Tpp1 should exacerbate the phenotype of the Cln3^{-/-} animal, or create a survival phenotype in Cln3 heterozygotes, but it is consistent with the possibility that CLN3 deficiency might have downstream effects on TPP1 function. An alternative possibility is that the compensatory increases in TPP1 activity detected in the absence of CLN3 might actually provide a neuroprotective function. Thus, heterozygosity for Tpp1 would diminish such a protective role, resulting in a shorter lifespan for the Cln3^{-/-} mouse.

Glial activation is a consistent pathological feature in NCL diseases, including both LINCL and JNCL. Typically, localized glial activation precedes the onset of neuron loss and serves to predict where neurodegeneration subsequently occurs. Indeed, there have been suggestions that glial dysfunction may contribute to neuron loss in multiple NCLs. The quantitative analysis of Tpp1^{-/-} Cln3^{+/+} and Tpp1^{-/-}Cln3^{-/-} mice revealed very similar extents of astrocytosis and microglial activation between mice of these genotypes.

Regardless of the underlying basis for its shortened lifespan, decreased survival of the Tpp1^{+/-}Cln3^{-/-} mutant may be useful for testing of therapeutic strategies for JNCL. In developing any therapeutic strategy, survival provides a clear and objective endpoint: for example, in evaluating several different strategies for LINCL, survival studies in mouse models have highlighted promising approaches (e.g., gene therapy, intrathecal cerebrospinal fluid (CSF)-mediated ERT, bloodstream mediated ERT) while survival studies in the dog model (Katz M L, et al. (2015) Sci Transl Med 7:313ra180) helped pave the way for the approval of enzyme replacement therapy. LINCL animal models have a markedly shortened lifespan; thus, proof of principle for treatments in terms of survival is readily achievable. In contrast, survival of Cln3 mutant mice approaches that of wild type, complicating survival as an endpoint. As a result, behavioral models have been extensively employed to analyze disease progression and the effects of potential treatment in Cln3 mutant mice. While numerous studies have characterized locomotor deficits in Cln3 mouse mutants, behavioral phenotypes are subtle and are dependent on mouse strain and gender.

Evaluation of potential therapeutics for JNCL in Cln3^{-/-}; Tpp1^{+/-} mouse mutants would provide a relatively robust survival phenotype to measure efficacy. In addition, it is likely that behavioral phenotypes would be exacerbated at a younger age. Effective therapy that addresses the loss of CLN3 (e.g., widespread gene therapy throughout the brain) would be predicted to increase survival of this mouse mutant to resemble that of the Tpp1 heterozygote, which is indistinguishable from wild type. A similar approach has been conducted using mutant Cln3 mice that express human amyloid precursor protein (APP) with familial Alzheimer disease mutations (Centa J L, et al. (2020) Nat Med 26:1444-1451). However, the mutant APP confers a severe survival phenotype even without CLN3 defects. Thus, positive treatment for CLN3 defects would essentially ameliorate a severe phenotype, not restore wild type survival.

Finally, one goal of this study was to determine whether the double Tpp1 and Cln3 knockout could highlight proteomic changes in the brain that could potentially provide a platform for clinically useful biomarkers in either or both LINCL and JNCL. Neurofilament light chain (NEFL in mice) has been proposed as a treatment-responsive biomarker based on studies of plasma from TPP1 patients and a dog model, and it was found levels of this, and other neurofilaments proteins (NEFH and NEFM) were elevated in mouse CSF. However, brain levels of NEFL, NEFH, and NEFM were unchanged or reduced in the TPP1 and CLN3 mouse models. This is consistent with the finding that brain NEFL is unchanged in mouse models of other neurodegenerative diseases exhibiting elevated NEFL plasma and CSF levels. It was also found that several lysosomal proteins (CTSF, SMPD1, and NPC1) were significantly altered in the Cln3^{-/-} animals, and while changes in expression were modest, they may be more evident in older mice (animals were analyzed at ˜120 days in this study). These changes may be compensatory responses to the loss of CLN3 and could potentially provide useful information regarding its biological function. In addition, soluble lysosomal proteins SMPD1 and CTSF may also warrant further investigation as potential biomarkersin INCL.

TABLE 1
Survival statistics for Tpp1^{-/-} animals. Survival data were obtained
from littermates from crosses between double Tpp1 and Cln3 mutant crosses
unless indicated otherwise (*for single mutant crosses). Significance
of the Log-rank tests conducted below was evaluated after correction
of P values for multiple comparisons using the Bonferroni method.

| | Total n/ | | | | |
|---|---|---|---|---|---|
| | number of | median | Log-rank (Mantel-Cox) test | | |
| Genotype | events | survival | P value | Compared to: | significant |
| Tpp1^{-/-}; Cln3^{-/-} | 204/134 | 120 | 0.0114 | Tpp1−/−; Cln3+/+ | no |
| Tpp1^{-/-}; Cln3^{+/-} | 126/108 | 124 | 0.5239 | Tpp1−/−; Cln3+/+ | no |
| Tpp1^{-/-}; Cln3^{+/+} | 97/45 | 124 | 0.8475 | Tpp1^{-/-}; Cln3^{+/+} | no |
| | | | | historical | |
| *Tpp1^{-/-}; Cln3^{+/+} | 3446/2242 | 125 | | | |
| historical | | | | | |
| *Tpp1^{+/+}; Cln3^{+/+} | 944/40 | nd | 0.4182 | Tpp1^{+/+}; Cln3^{+/+} | no |
| | | | | (Yuan dataset) | |
| Tpp1^{+/+}; Cln3^{+/+} (Yuan | 61/61 | 901 | | | |
| dataset) | | | | | |
| Tpp1^{+/-}; Cln3^{-/-} | 85/15 | 584 | 0.0001 | Tpp1+/+; Cln3−/− | yes |
| Tpp1^{+/-}; Cln3^{+/-} | 248/18 | 748 | 0.7674 | Tpp1+/+; Cln3−/− | no |
| | | | 0.0014 | Tpp1+/+; Cln3+/+ | yes |
| | | | | (Yuan dataset) | |
| *Tpp1^{+/-}; Cln3^{+/+} | 1356/47 | 843 | 0.1072 | Tpp1+/+; Cln3+/+ | no |
| | | | | (Yuan dataset) | |
| *Tpp1^{+/+}; Cln3^{-/-} | 254/23 | 719 | <0.0001 | Tpp1+/+; Cln3+/+ | yes |
| | | | | (Yuan dataset) | |
| Tpp1^{+/+}; Cln3^{-/-} (Katz | 27/27 | 742 | 0.3112 | Tpp1^{+/+}; Cln3^{-/-} | no |
| dataset) | | | | | |

TABLE 2
Comparison of proteomic changes in Tpp1-mutant mouse models
compared to wild type. Compared to wild type, proteins shown
are significantly altered in at least one model (FDR 1%), have
a magnitude of change of ≥1.5 fold, and have a consistent
direction of change in both models. Significant changes are
indicated in bold. Note that NNT is censored (see Examples).

| | | |
|---|---|---|
| | Ratio | Ratio |
| | Tpp1^{-/-}Cln3^{+/+} | Tpp1^{-/-}Cln3^{-/-} |
| Protein | v Tpp1^{+/+}Cln3^{+/+} | v Tpp1^{+/+}Cln3^{+/+} |

lysosomal

| | | |
|---|---|---|
| NPC1 | 1.45 | 2.46 |
| CTSH | 1.88 | 1.88 |
| CD63 | 1.42 | 1.78 |
| LAMP1 | 1.35 | 1.61 |
| CTSZ | 1.60 | 1.60 |
| CTSS | 1.99 | 1.60 |
| FUCA1 | 1.30 | 1.59 |
| CTSD | 1.62 | 1.42 |

non-lysosomal

| | | |
|---|---|---|
| GPNMB | 4.99 | 4.61 |
| WDFY1 | 2.20 | 4.49 |
| LYZ2 | 3.68 | 4.28 |
| A2M | 2.53 | 3.36 |
| LGALS3 | 2.29 | 2.75 |
| RNF13 | 1.35 | 1.99 |
| LGALS3BP | 2.90 | 1.90 |
| SERPINA3N | 2.12 | 1.83 |
| ATP2C1 | 1.11 | 1.74 |
| TAPBP | 1.90 | 1.57 |
| CD44 | 1.47 | 1.56 |
| IFIT3 | 1.84 | 1.41 |
| SGTB | 0.64 | 0.84 |
| PRPH | 0.58 | 0.84 |
| VIP | 1.01 | 0.65 |
| COX6C | 0.90 | 0.64 |
| MCEE | 0.86 | 0.63 |
| RPL27-PS3 | 0.86 | 0.63 |
| IGSF5 | 0.70 | 0.61 |
| BCL11B | 0.81 | 0.53 |
| KRT42 | 0.70 | 0.43 |
| IFRD1 | 1.08 | 0.06 |
| SLC47A1 | 0.27 | 0.06 |
| C1QA | 2.62 | 1.92 |
| MFSD1 | 1.28 | 1.80 |
| H2AFZ | 0.42 | 1.35 |
| GBP10 | 1.77 | 1.34 |
| IRGM1 | 1.52 | 1.28 |
| ISG15 | 2.05 | 1.27 |
| CLDN11 | 1.52 | 1.15 |
| SQSTM1 | 1.59 | 1.12 |

### Example 3

One of the goals of this study was to determine whether overexpression of TPP1 could reduce storage of Subunit C mitochondrial ATP synthase (SCMAS) in JNCL mice. A mouse transgenic model was previously developed, which constitutively over-expresses TPP1 (R26Tg^{TPP1+/0}) (Nemtsova, Y., et al., PLoS One, 2018. 13(2): p. e0192286). It allowed us to test this hypothesis using a genetic approach.

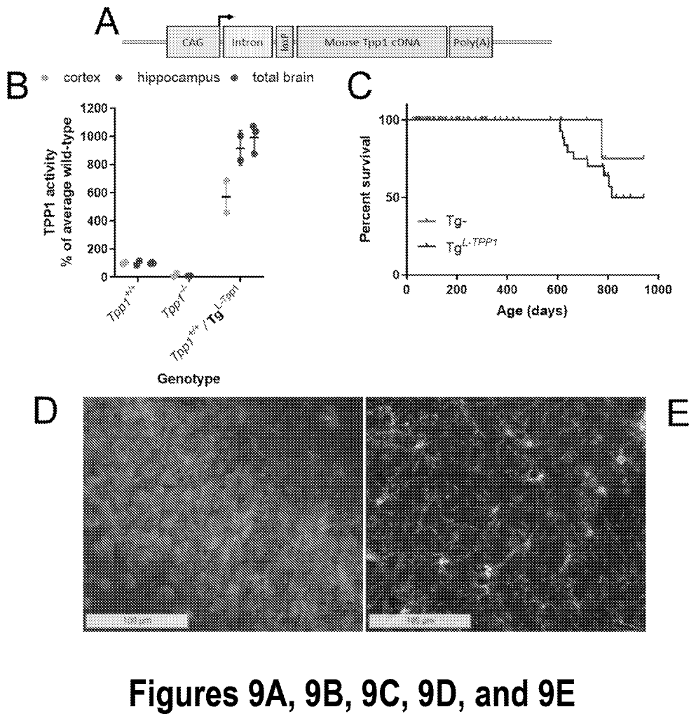

The transgenic overexpresses murine TPP1 from a CAG-promoter driven transgene (Tg^{TPP1+}) integrated at the ROSA26 locus (FIG. 9A). This mouse expresses TPP1 at levels at least 10-fold higher than normal in brain (FIG. 9B). TPP1 overexpression is constitutive and ubiquitous throughout the brain, including cortex and hippocampus (FIG. 9B). TPP1 overexpression has no apparent deleterious effects on the animals (FIG. 9C). Transgenic TPP1 is expressed in both neurons and microglia, colocalizing with respective markers NeuN (FIG. 9D) and Iba1 (FIG. 9E).

The approach was to cross our TPP1-overexpressing transgenic with the Cln3^{-/-} JNCL mouse model to determine whether elevated TPP1 levels have any positive effect on disease progression. Four experimental cohorts of littermate mice were established: 1) Tg-Cln3^{+/+}; 2) Tg-Cln3^{-/-}; 3) Tg+Cln3^{+/+} and; 4) Tg+Cin3^{-/-}. SCMAS storage in liver and spleen of the JNCL mice at 6 months of age were examined. SCMAS accumulation in brain at this time-point was also examined.

### Methods

Animals. Animals were anesthetized with Euthasol (pentobarbital sodium and phenytoin sodium) and then euthanized by exsanguination/transcardial perfusion with PBS and dissected.

Half of the brain was rapidly frozen for biochemical analyses (see below) and the other half drop-fixed in 4% paraformaldehyde (PFA) for immunohistochemistry.

### Histology Embedding, Sectioning & Staining

Immunohistochemistry was performed by NeuroScience Associates (Knoxville, TN). Tissue samples (brain, liver spleen) were treated overnight with 20% glycerol and 2% dimethylsulfoxide. Samples were then embedded in a gelatin matrix using MultiBrain®/MultiCord® Technology. Blocks were rapidly frozen, after curing by immersion in 2-Methylbutane chilled with crushed dry ice and mounted on a freezing stage of an AO 860 sliding microtome and blocks were sectioned. All sections were cut through the entire length of the specimen segment and collected sequentially into series of 24 containers containing Antigen Preserve solution (50% PBS pH7.0, 50% Ethylene Glycol, 1% Polyvinyl Pyrrolidone).

### Immunohistochemistry

Lysosomal storage of SCMAS was visualized using an affinity-purified rabbit anti-polyclonal antibody raised against SCMAS amino-terminal peptide (PAC3601/3602; Pacific Immunology, Ramona, CA) (Xu, S., et al., Mol Ther, 2011. 19(10): p. 1842-8). Free floating sections were stained with desired stain. All incubation solutions from the primary antibody onward use Tris buffered saline (TBS) with Triton X-100 as the vehicle; all rinses are with TBS. After a hydrogen peroxide treatment, free floating sections were immunostained with the anti-SCMAS antibody overnight at room temperature at a dilution of 1:500. Vehicle solutions contained Triton X-100 for permeabilization. Following rinses, a biotinylated secondary antibody (Anti-Rabbit IgG, made in goat from Vector Labs) was applied at a dilution of 1:1000. After further rinses Vector Lab's ABC solution (avidin-biotin-HRP complex; details in instruction for VECTASTAIN® Elite ABC, Vector, Burlingame, CA) was applied. The sections were again rinsed, then treated with diaminobenzidine tetrahydrochloride (DAB) with nickel and hydrogen peroxide to create a visible reaction product. Following further rinses, the sections were mounted on gelatin coated glass slides, air dried. The slides were dehydrated in alcohols, cleared in xylene and coverslipped.

### Imaging

Slides were scanned using a TissueScope LE system from Huron Digital Pathology. Whole slides were scanned at 20× resolution (0.4 um/pixel). SCMAS accumulation was quantified using ImageJ (Schneider, C.A., et al. Nat Methods, 2012. 9(7): p. 671-5.).

### Results

Animals were euthanized at 6 months of age and SCMAS accumulation evaluated in spleen, liver and brain.

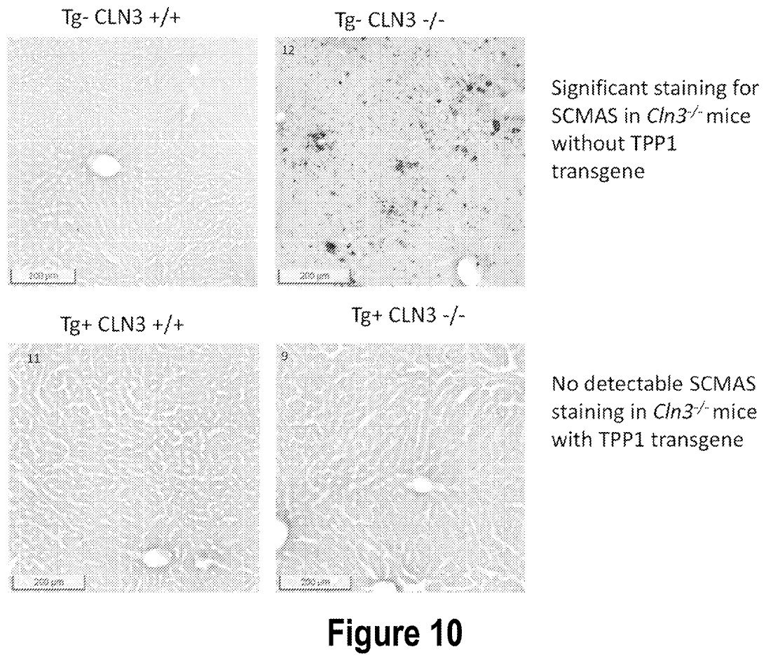

Liver. No SCMAS staining was detected in liver sections from control animals (Tg-Cln3^{+/+} and Tg+ Cln3^{+/+}) (FIG. 10). In Cln3^{-/-} mice lacking the TPP1 transgene (Tg− Cln3^{-/-}), extensive storage of SCMAS was detected in the liver. In Cln3^{-/-} mice expressing the Tpp1 transgene (Tg+ Cln3^{-/-}), SCMAS storage was not detected.

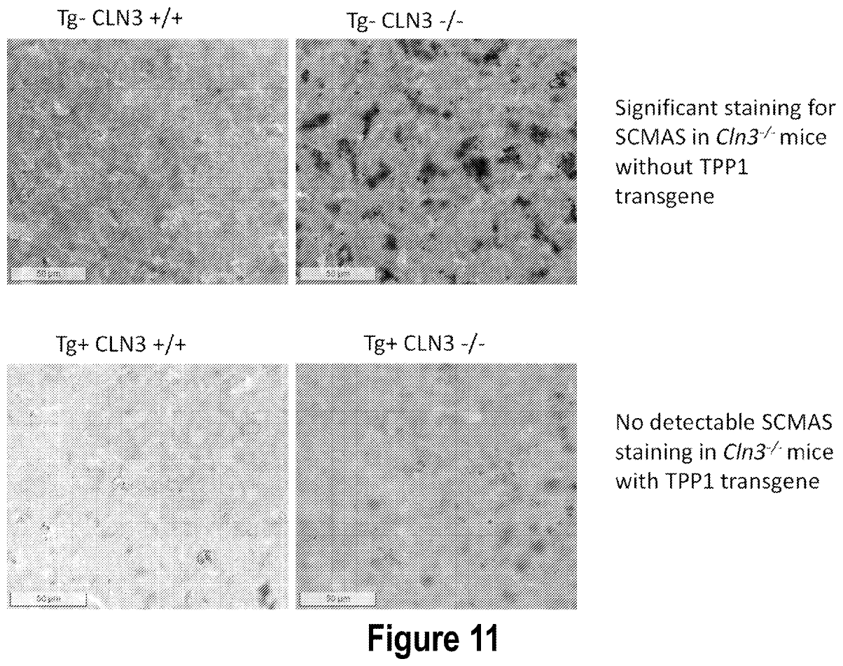

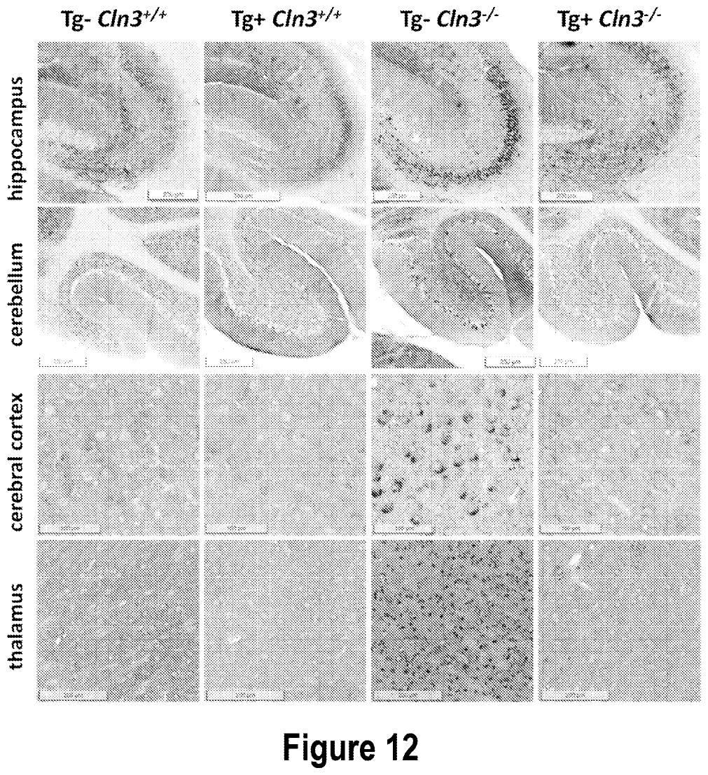

Spleen. No SCMAS staining was detected in spleen sections from control animals (Tg− Cln3^{+/+} and Tg+ Cln3^{+/+}) (FIG. 11). In Cln3^{-/-} mice lacking the TPP1 transgene (Tg−Cln3^{-/-}), extensive storage of SCMAS was detected in the spleen. In Cln3^{-/-} mice expressing the Tpp1 transgene (Tg+ Cln3−/−), SCMAS storage was not detected.

Brain. SCMAS storage was analyzed in brain in 4 different regions: CA3 region of hippocampus, cerebellum, thalamus and cortex. No SCMAS staining was detected in any brain regions sections from control animals (Tg− Cln3^{+/+} and Tg+ Cln3^{+/+}) (FIG. 12). In Cln3^{-/-} mice lacking the TPP1 transgene (Tg− Cln3^{-/-}), extensive storage of SCMAS was detected specifically in the CA3 region of the hippocampus and in the Purkinje cells of the cerebellum. Widespread storage was observed in the thalamus and cortex. In JNCL mice expressing the TPP1 transgene (Tg+ Cln3^{-/-}), SCMAS storage was not detected in any of these regions.

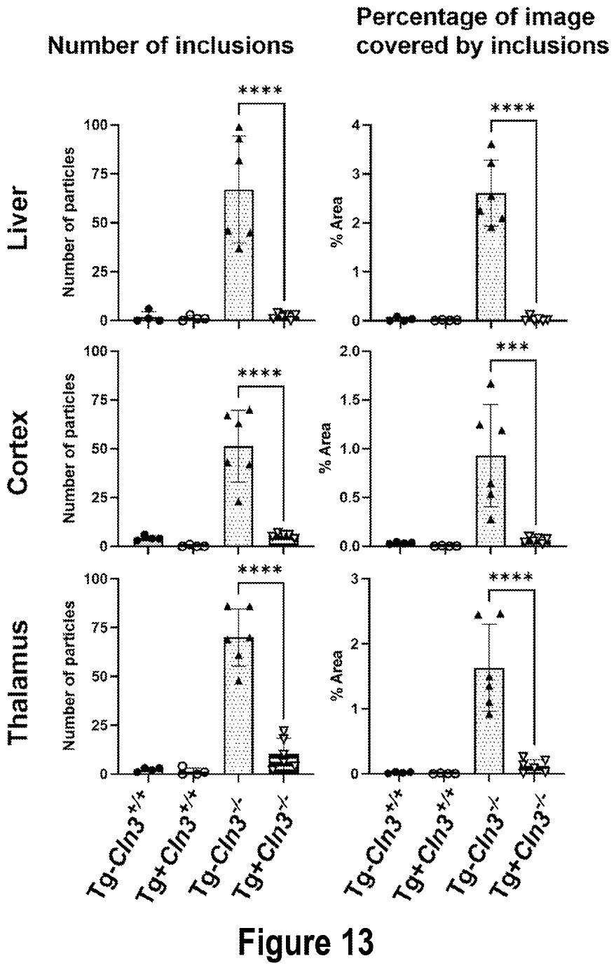

SCMAS storage in liver, cortex and thalamus was quantified in terms of area of image occluded by inclusions (FIG. 13). The effect of transgene expression on storage of SCMAS is clear.

JNCL mice expressing the TPP1 transgene (Tg+ Cln3^{-/-}) showed no detectable accumulation of SCMAS in liver, spleen or four regions of brain at 6 months of age. These results indicate that elevated TPP1 activity can prevent the appearance of storage material in JNCL and provide support for TPP1 augmentation as a potential therapeutic approach in JNCL and other diseases that may accumulate SCMAS.

The present disclosure is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

What is claimed is:

1. A method of treating a subject having a disease or disorder characterized by accumulation of SCMAS in the lysosomes of affected cells, comprising administering to the subject a therapeutically effective amount of an agent that increases a level or activity of TPP1, to reduce or eliminate symptoms caused by the disease or disorder.

2. The method of claim 1, wherein the disease or disorder is selected from Juvenile neuronal ceroid lipofuscinosis (CLN3) disease, Variant late infantile neuronal ceroid lipofuscinosis type 5 (CLN5) disease, Variant late infantile neuronal ceroid lipofuscinosis type 6 (CLN6) disease, Neuronal ceroid lipofuscinosis type 7 (CLN7) disease, Northern epilepsy neuronal ceroid lipofuscinosis type 8 (CLN8) disease, Congenital neuronal ceroid lipofuscinosis type 10 (CLN10) disease, Late-onset Neuronal ceroid lipofuscinosis (CLN12) and Kufor-Rakeb syndrome, Sanfilippo D syndrome (mucopolysaccharidosis type HID), and Osteopetrosis autosomal recessive 4 (OPTB4).

3. The method of any one of the preceding claims, wherein the disease or disorder is characterized by a deficiency in a function of a CLN3 protein.

4. The method of any one of the preceding claims, wherein the agent reduces the level of accumulation of the SCMAS in the lysosomes of the affected cells.

5. The method of any one of the preceding claims, wherein the affected cells are neuronal cells.

6. The method of any one of the preceding claims, wherein the agent comprises a recombinant human TPP1 protein or a nucleic acid molecule comprising a nucleotide sequence encoding TPP1 or a variant thereof.

7. The method of claim 6, wherein the TPP1 protein is an inactive proenzyme.

8. The method of any one of claims 6 to 7, wherein the TPP1 protein is mannose-6-phosphorylated.

9. The method of any one of claims 6 to 8, wherein the therapeutically effective amount of the TPP1 protein is such that the affected cells receive from about 1.0 to about 100 nM of recombinant human TPP1 protein.

10. The method of any one of claims 6 to 9, wherein the agent is administered by injection.

11. The method of any one of claim 10, wherein the injection is intracranial.

12. The method of any one of the preceding claims, wherein the agent is administered in a controlled release system.

13. The method of any one of the preceding claims, wherein the agent is delivered to lysosomes of the affected cells.

14. The method of any one of the preceding claims, wherein the subject is a human.

15. An animal model for studying a disease or disorder, comprising: (i) a tripeptidyl peptidase 1 (Tpp1) gene heterozygous knockout (Tpp1^{+/-}); and (ii) a Cln3 gene homozygous knockout (Cln3^{-/-}), wherein the mouse model has a shortened lifespan compared to a wild type animal.

16. The animal model of claim 15, wherein the animal is a mouse.

17. The animal model of any one of claims 15 to 16, wherein the disease or disorder is selected from Late-infantile neuronal ceroid lipofuscinosis (CLN2) disease, Juvenile neuronal ceroid lipofuscinosis (CLN3) disease, Variant late infantile neuronal ceroid lipofuscinosis type 5 (CLN5) disease, Variant late infantile neuronal ceroid lipofuscinosis type 6 (CLN6) disease, Neuronal ceroid lipofuscinosis type 7 (CLN7) disease, Northern epilepsy neuronal ceroid lipofuscinosis type 8 (CLN8) disease, Congenital neuronal ceroid lipofuscinosis type 10 (CLN10) disease, Late-onset Neuronal ceroid lipofuscinosis (CLN12) and Kufor-Rakeb syndrome, Sanfilippo D syndrome (mucopolysaccharidosis type HID), and Osteopetrosis autosomal recessive 4 (OPTB4).

18. The animal model of any one of claims 15 to 17, wherein the animal has at least 25% reduction in lifespan compared to the wild type animal.

19. The animal model of any one of claims 15 to 18, wherein the Tpp1 gene or the Cln3 gene comprises at least one mutation selected from a deletion, an insertion, a frame-shift mutation, re-arrangement or a substitution.

20. The animal model of claim 19, wherein the mutation is constitutive.

21. The animal model of claim 19, wherein the mutation is conditional.

22. The animal model of any one of the preceding claims, wherein the Tpp1 gene comprises a deletion of at least a portion of an exon within the Tpp1 gene.

23. The animal model of any one of claims 15 to 22, wherein the Tpp1 gene comprises an insertion of neo into intron 11 and an Arg446His missense mutation into exon 11 immediately upstream of the neo insertion.

24. The animal model of any one of claims 15 to 23, wherein the Tpp1 gene is located at Chr 7 E3; 7 55.97 cM.

25. The animal model of any one of claims 15 to 24, wherein the Cln3 gene comprises a deletion of at least a portion of an exon within the Cln3 gene.

26. The animal model of any one of claims 15 to 25, wherein the Cln3 gene comprises a deletion of all or part of exons 1-6 within the Cln3 gene.

27. The animal model of any one of claims 15 to 26, wherein the Cln3 gene is located at Chr 7 F3; 7 69.16 cM.

28. The animal model of any one of claims 15 to 27, wherein the animal model has an increased level of lysosomal accumulation of subunit c of mitochondrial ATP synthase (SCMAS).

29. The animal model of claim 28, wherein the animal model has at least 50% increase in the level of lysosomal accumulation of SCMAS.

30. The animal model of any one of claims 15 to 29, wherein the animal model has an increased expression level of Niemann-Pick disease type C1 (NPC1) and/or Cathepsin F (CTSF) or reduced expression of SMPD1.

31. The animal model of claim 30, wherein the animal model has at least 40% increase in the expression level of NPC1 and/or CTSF or 40% decrease in the expression level of acid sphingomyelinase (SMPD1).

32. The animal model of any one of claims 15 to 31, wherein the animal model is characterized by a deficit in a locomotor activity.

33. A progeny of the animal model of any one of claims 15 to 32.

34. A cell, tissue, or cell line derived from the animal model of any one of claims 15 to 32 or the progeny of claim 33.

35. A method of obtaining the animal model of any one of claims 15 to 32, comprising: (a) cross-breeding an animal with a Tpp1 knockout with a second animal with a Cln3 knockout to obtain an animal with double heterozygotes (Tpp1^{+/-}; Cln3^{+/-}); and (b) cross-breeding the animal with double heterozygotes by mating Tpp1^{-/-}; Cln3^{-/-}×Tpp1^{-/-}; Cln3^{-/-} or Tpp1^{-/-}; Cln3^{-/+}×Tpp1^{+/-}; Cln3^{-/-}.

36. A method of identifying an agent for use in treatment of a disease or disorder in a subject, comprising:
(i) administering a candidate agent to the animal model of any one of claims 15 to 32 or the progeny of claim 33, and assessing an effect of the candidate agent on a phenotype of the animal model; or
(ii) contacting the cell, tissue, or cell line of claim 34 with a candidate agent, and assessing an effect of the candidate agent on the cell, tissue, or cell line.

37. The method of claim 36, wherein the disease or disorder is characterized by accumulation of SCMAS in the lysosomes of affected cells of the animal model.

38. The method of any one of claims 36 to 37, wherein the disease or disorder is selected from Late-infantile neuronal ceroid lipofuscinosis (CLN2) disease, Juvenile neuronal ceroid lipofuscinosis (CLN3) disease, Variant late infantile neuronal ceroid lipofuscinosis type 5 (CLN5) disease, Variant late infantile neuronal ceroid lipofuscinosis type 6 (CLN6) disease, Neuronal ceroid lipofuscinosis type 7 (CLN7) disease, Northern epilepsy neuronal ceroid lipofuscinosis type 8 (CLN8) disease, Congenital neuronal ceroid lipofuscinosis type 10 (CLN10) disease, Late-onset Neuronal ceroid lipofuscinosis (CLN12) and Kufor-Rakeb syndrome, Sanfilippo D syndrome (mucopolysaccharidosis type HID), and Osteopetrosis autosomal recessive 4 (OPTB4).

39. The method of claims 36 to 38, wherein the phenotype is a lifespan of the animal model.

40. The method of any one of claims 36 to 39, wherein the effect is characterized by an increase in the lifespan of the animal model.

41. The method of any one of claims 36 to 40, wherein the effect is characterized by a decrease in the level of lysosomal accumulation of SCMAS.

42. The method of any one of claims 36 to 41, wherein the effect is characterized by a decrease in the expression level of NPC1 and/or CTSF.

43. The method of any one of claims 36 to 42, wherein the candidate agent comprises a protein, a peptide, a peptidomimetic, a nucleic acid, or a small molecule.
